SEQUENCE LISTING (41 sequences)

SEQ ID NO: 1 - RNA, 84 nt, Homo sapiens
  source 1..84: mol_type = other RNA
gtcagaataatgtcaaagtgcttacagtgcaggtagtgatatgtgcatctactgcagtgaaggcacttgtagcattatggtgac

SEQ ID NO: 2 - RNA, 22 nt, Homo sapiens
  source 1..22: mol_type = other RNA
actgcagtgaaggcacttgtag

SEQ ID NO: 3 - RNA, 23 nt, Homo sapiens
  source 1..23: mol_type = other RNA
caaagtgcttacagtgcaggtag

SEQ ID NO: 4 - RNA, 95 nt, Homo sapiens
  source 1..95: mol_type = other RNA
ttgtacctggtgtgattataaagcaatgagactgattgtcatatgtcgtttgtgggatccgtctcagttactttatagccatacctggtatctta

SEQ ID NO: 5 - RNA, 22 nt, Homo sapiens
  source 1..22: mol_type = other RNA
ttataaagcaatgagactgatt

SEQ ID NO: 6 - RNA, 22 nt, Homo sapiens
  source 1..22: mol_type = other RNA
tccgtctcagttactttatagc

SEQ ID NO: 7 - RNA, 110 nt, Homo sapiens
  source 1..110: mol_type = other RNA
gctgctggaaggtgtaggtaccctcaatggctcagtagccagtgtagatcctgtctttcgtaatcagcagctacatctggctactgggtctctgatggcatcttctagct

SEQ ID NO: 8 - RNA, 22 nt, Homo sapiens
  source 1..22: mol_type = other RNA
ctcagtagccagtgtagatcct

SEQ ID NO: 9 - RNA, 21 nt, Homo sapiens
  source 1..21: mol_type = other RNA
agctacatctggctactgggt

SEQ ID NO: 10 - RNA, 110 nt, Homo sapiens
  source 1..110: mol_type = other RNA
tgaacatccaggtctggggcatgaacctggcatacaatgtagatttctgtgttcgttaggcaacagctacattgtctgctgggtttcaggctacctggaaacatgttctc

SEQ ID NO: 11 - RNA, 22 nt, Homo sapiens
  source 1..22: mol_type = other RNA
acctggcatacaatgtagattt

SEQ ID NO: 12 - RNA, 23 nt, Homo sapiens
  source 1..23: mol_type = other RNA
agctacattgtctgctgggtttc

SEQ ID NO: 13 - RNA, 96 nt, Homo sapiens
  source 1..96: mol_type = other RNA
agatgtgctctcctggcccatgaaatcaagcgtgggtgagacctggtgcagaacgggaaggcgacccatacttggtttcagaggctgtgagaataa

SEQ ID NO: 14 - RNA, 22 nt, Homo sapiens
  source 1..22: mol_type = other RNA
gaaatcaagcgtgggtgagacc

SEQ ID NO: 15 - RNA, 21 nt, Homo sapiens
  source 1..21: mol_type = other RNA
gcgacccatacttggtttcag

SEQ ID NO: 16 - DNA, 20 bp, synthetic construct
  misc_feature 1..20: note = primer VIM forward
  source 1..20: mol_type = other DNA
gacgccatcaacaccgagtt

SEQ ID NO: 17 - DNA, 21 bp, synthetic construct
  misc_feature 1..21: note = Primer VIM Reverse
  source 1..21: mol_type = other DNA
ctttgtcgttggttagctggt

SEQ ID NO: 18 - DNA, 21 bp, synthetic construct
  misc_feature 1..21: note = Primer RAP2A Froward
  source 1..21: mol_type = other DNA
cgtgaagcggtatgagaaagt

SEQ ID NO: 19 - DNA, 19 bp, synthetic construct
  misc_feature 1..19: note = Primer RAP2A Reverse
  source 1..19: mol_type = other DNA
gccttcgctggacgatact

SEQ ID NO: 20 - DNA, 21 bp, synthetic construct
  misc_feature 1..21: note = Primer LITAF Forward
  source 1..21: mol_type = other DNA
atgtcggttccaggaccttac

SEQ ID NO: 21 - DNA, 21 bp, synthetic construct
  misc_feature 1..21: note = Primer LITAF Reverse
  source 1..21: mol_type = other DNA
tacgaaggaggattcatgccc

SEQ ID NO: 22 - DNA, 21 bp, synthetic construct
  misc_feature 1..21: note = Primer NFKBIZ Forward
  source 1..21: mol_type = other DNA
acacccacaaaccaactctgg

SEQ ID NO: 23 - DNA, 21 bp, synthetic construct
  misc_feature 1..21: note = Primer NFKBIZ Reverse
  source 1..21: mol_type = other DNA
ggaaaactgtgattctggacc

SEQ ID NO: 24 - DNA, 20 bp, synthetic construct
  misc_feature 1..20: note = Primer ROCK1 Forward
  source 1..20: mol_type = other DNA
aaccatgtgactgagtgccc

SEQ ID NO: 25 - DNA, 20 bp, synthetic construct
  misc_feature 1..20: note = Primer ROCK1 Reverse
  source 1..20: mol_type = other DNA
tcagtgtgttgtgccaaagc

SEQ ID NO: 26 - DNA, 20 bp, synthetic construct
  misc_feature 1..20: note = Primer CHRNA4 Forward
  source 1..20: mol_type = other DNA
ggagggcgtccagtacattg

SEQ ID NO: 27 - DNA, 20 bp, synthetic construct
  misc_feature 1..20: note = Primer CHRNA4 Reverse
  source 1..20: mol_type = other DNA
gaagatgcggtcgatgacca

SEQ ID NO: 28 - DNA, 20 bp, synthetic construct
  misc_feature 1..20: note = Primer BDKRB2 Forward
  source 1..20: mol_type = other DNA
gtctgttcgtgaggactccg

SEQ ID NO: 29 - DNA, 21 bp, synthetic construct
  misc_feature 1..21: note = Primer BDKRB2 Reverse
  source 1..21: mol_type = other DNA
aaaggtcccgttaagagtggg

SEQ ID NO: 30 - DNA, 20 bp, synthetic construct
  misc_feature 1..20: note = Primer GALNT3 Forward
  source 1..20: mol_type = other DNA
tcttggaccagacactcgac

SEQ ID NO: 31 - DNA, 21 bp, synthetic construct
  misc_feature 1..21: note = Primer GALNT3 Reverse
  source 1..21: mol_type = other DNA
ggacagttctaagcaacgtcc

SEQ ID NO: 32 - DNA, 20 bp, synthetic construct
  misc_feature 1..20: note = Primer STMN3 Forward
  source 1..20: mol_type = other DNA
cgcaccccaataccgtctac

SEQ ID NO: 33 - DNA, 21 bp, synthetic construct
  misc_feature 1..21: note = Primer STMN3 Reverse
  source 1..21: mol_type = other DNA
ggacaggtcagaaggggactt

SEQ ID NO: 34 - DNA, 20 bp, synthetic construct
  misc_feature 1..20: note = Primer COL5A3 Forward
  source 1..20: mol_type = other DNA
gtggccgtcagcatagatgg

SEQ ID NO: 35 - DNA, 23 bp, synthetic construct
  misc_feature 1..23: note = Primer COL5A3 Reverse
  source 1..23: mol_type = other DNA
tgaatgtctccctcgaaagtctt

SEQ ID NO: 36 - DNA, 21 bp, synthetic construct
  misc_feature 1..21: note = Primer EEF1A1 Forward
  source 1..21: mol_type = other DNA
agcaaaaatgacccaccaatg

SEQ ID NO: 37 - DNA, 20 bp, synthetic construct
  misc_feature 1..20: note = Primer EEF1A1 Reverse
  source 1..20: mol_type = other DNA
ggcctggatggttcaggata

SEQ ID NO: 38 - DNA, 19 bp, synthetic construct
  misc_feature 1..19: note = Primer ALAS1 Forward
  source 1..19: mol_type = other DNA
ctcaccacacacccagatg

SEQ ID NO: 39 - DNA, 18 bp, synthetic construct
  misc_feature 1..18: note = Primer ALAS1 Reverse
  source 1..18: mol_type = other DNA
agttccagcccacttgct

SEQ ID NO: 40 - RNA, 22 nt, Homo sapiens
  source 1..22: mol_type = other RNA
tagcagcacgtaaatattggcg

SEQ ID NO: 41 - RNA, 22 nt, Homo sapiens
  source 1..22: mol_type = other RNA
caacggaatcccaaaacagctg

(19) United States
(12) Patent Application Publication
(10) Pub. No.: US 2025/0283092 A1
(43) Pub. Date: Sep. 11, 2025

(54) MIRNA COMBINATION FOR THE PREVENTION AND TREATMENT OF CANCER

(71) Applicants: Centre Leon Berard, Lyon (FR); Universite Claude Bernard Lyon 1, Villeurbanne (FR); Centre National de la Recherche Scientifique, Paris (FR); Institut National de la Santé et de la Recherche Medicale, Paris (FR)

(72) Inventor: Erika Cosset, Chens-Sur-Leman (FR)

(21) Appl. No.: 18/696,878

(22) PCT Filed: Sep. 27, 2022
(86) PCT No.: PCT/EP2022/076873

§ 371 Date: Mar. 28, 2024

(30) Foreign Application Priority Data
Sep. 28, 2021 (EP) 21306343.1

(51) Int. Cl. C12N 15/113; A61P 35/00

(52) U.S. Cl. CPC C12N 15/1138; A61P 35/00; C12N 15/113; C12N 15/1137; C12N 2310/113; C12N 2310/141; C12N 2320/31

(57) ABSTRACT

The present invention relates to the combination of a miR-17mimic and a miR-340 mimic for use for the prevention and/or treatment of cancer, in particular for the treatment of glioblastoma. In particular, said combination additionally comprises a miR-222 antagomiR.

The present invention relates to the combination of a miR-17mimic and a miR-340 mimic for use for the prevention and/or treatment of cancer, in particular for the treatment of glioblastoma. In particular, said combination additionally comprises a miR-222 antagomiR.

Glioblastoma (GBM) is a grade IV astrocytoma, a deadly malignant brain tumor and among the most common primary brain tumor in adults. Today, surgery, radiotherapy, and chemotherapy with temozolomide remain the standard of care in patients with GBM (Stupp, et al. (2005). Radiotherapy plus concomitant and adjuvant temozolomide for glioblastoma. N Engl J Med 352: 987-996). However, the median overall survival of patients with GBM (around 14 months) has not radically changed over the last 15 years. Major efforts in large-scale genomic and transcriptomic profiling allow the characterization and stratification of GBM patients into four subtypes: Classical, Mesenchymal, Proneural, and Neural (which has been recently removed), (Verhaak, R G et al. (2010). Integrated genomic analysis identifies clinically relevant subtypes of glioblastoma characterized by abnormalities in PDGFRA, IDH1, EGFR, and NF1. Cancer Cell 17: 98-110.; Brennan, C W, et al. (2013). The somatic genomic landscape of glioblastoma. Cell 155: 462-477.; Freije, W A et al. (2004). Gene expression profiling of gliomas strongly predicts survival. Cancer Res 64: 6503-6510). Nevertheless, these big data analyses have yet to highlight new therapeutic avenues and druggable molecules to achieve advances in precision medicine.

MiRNAs are small non-coding RNAs consisting of 20 to 22 nucleotides that participate in the posttranslational regulation of gene expression through RNA interference processes (Bartel, D P, et al. (2004). Micromanagers of gene expression: the potentially widespread influence of metazoan microRNAs. Nat Rev Genet 5: 396-400.; Lee, Y, et al. (2002). MicroRNA maturation: stepwise processing and subcellular localization. EMBO J 21: 4663-4670.; Tomari, Y, et al. (2005). MicroRNA biogenesis: drosha can't cut it without a partner. Curr Biol 15: R61-64). The miRNA genes are transcribed by RNA polymerase II to form pri-miRNA transcripts. These pri-miRNA are processed by Drosha (class 2 RNAse III enzyme) to release the pre-miRNA precursor product consisting of approximately 70 nucleotides. Finally, the pre-miRNA is exported to the cytoplasm where it is processed to generate a mature of around 20-nucleotide miRNA. This miRNA is integrated into the RISC complex (an endoribonuclease of RNase III family) and forms double-stranded RNA when binding the complementary target mRNAs. As a consequence, a cleavage at the loop-end of the miRNA structure generates the 5p and 3p strands, where 5p and 3p define whether the miRNA originates from the 5′ or 3′ end of a pre-miRNA hairpin, respectively. Based on genomic and functional experiments, miRNA activity in humans was mainly attributed to 5p strands. Indeed, 3p strands are much less abundant in RISC and are rapidly removed. Depending on miRNAs' complementation with the target mRNAs, the RISC complex inhibits mRNA translation or induces mRNA degradation (Tomari, Y, et al. (2005). MicroRNA biogenesis: drosha can't cut it without a partner. Curr Biol 15: R61-64.; Zhang, H, et al. (2004). Single processing center models for human Dicer and bacterial RNase III. Cell 118: 57-68). Consistent with recent computational predictions, each miRNA has the potential to regulate about 200 target genes; thus miRNA-mediated gene regulation is now considered to have an important role in biologic processes (Lewis, B P, et al. (2005). Conserved seed pairing, often flanked by adenosines, indicates that thousands of human genes are microRNA targets. Cell 120: 15-20).

MiRNAs undergo aberrant expression during tumorigenesis and miRNAs-coding genes are frequently located at fragile sites, in regions gained and lost in mammalian cancers (Calin, G A, et al. (2004). Human microRNA genes are frequently located at fragile sites and genomic regions involved in cancers. Proc Natl Acad Sci USA 101: 2999-3004).

Because they play critical roles in various vitally important cellular processes, the use of miRNAs in personalized cancer therapy is extremely attractive.

However, more investigation is clearly needed to identify new drivers of GBM aggressiveness and clinically and biologically relevant miRNAs for GBM therapeutic strategies.

The inventors of the present invention have identified miR-17 and miR-340 as clinically relevant miRNAs that can be used for a GBM multitargeting therapeutic strategy. They found that the ectopic expression of miR-17 and miR-340 inhibited tumor aggressiveness in vitro and in vivo.

They also found that the combination of miR-17, miR-340 and miR-222 shows a systematic and significant inhibition of tumor growth in all subtypes of GBM, in vitro and in vivo.

The present invention thus relates to a combination of a miR-17mimic and a miR-340 mimic for use for the prevention and/or treatment of cancer.

In particular, said combination additionally comprises a miR-222 antagomiR.

More particularly, said combination additionally comprises a miR-221 antagomiR and/or a miR-551 bmimic.

The term “combination” as used herein, refers to a composition comprising a plurality of components. The use of the term “combination” does not restrict the relative contents of the different components of the composition, which may be found in equimolar ratios, in the same weight ration or in different molar or weight ratios.

As a consequence, the following combinations are within the scope of the present invention:
combination of a miR-17mimic and a miR-340 mimic;
combination of a miR-17mimic, a miR-340 mimic and a miR-222 antagomiR;
combination of a miR-17mimic, a miR-340 mimic and a miR-221 antagomiR;
combination of a miR-17mimic, a miR-340 mimic, a miR-222 antagomiR and a miR-221 antagomiR;
combination of a miR-17mimic, a miR-340 mimic and a miR-551b mimic;
combination of a miR-17mimic, a miR-340 mimic, a miR-222 antagomiR and a miR-551b mimic;
combination of a miR-17mimic, a miR-340 mimic, a miR-221 antagomiR and a miR-551b mimic; and
combination of a miR-17mimic, a miR-340 mimic, a miR-222 antagomiR, a miR-221 antogomiR and a miR-551 b-mimic.

As mentioned above, miRNAs are small non-coding RNAs consisting of 20 to 22 nucleotides that participate in the posttranslational regulation of gene expression through RNA interference processes.

The terms “microRNA” or “miRNA” or “miR” are used herein interchangeably to refer to a small non-coding RNA, which functions in transcriptional and/or post-transcriptional regulation of gene expression. In various embodiments, miRNAs have a hairpin structure comprising a duplex that is processed into a guide strand and a passenger strand.

As used herein, the term “miR” encompasses any isoform of the said miRNA and all members of the said miRNA family. The term “miRNA” thus includes star-sequences and family members. In particular, miRNAs according to the invention are human i.e. hsa-miRNA.

MiRNA nomenclature remains inconsistent. However, lettered suffixes are used to name genes encoding miRNA sisters (for example miR-17a and miR17b). Then, numeric suffixes are used at the end of miRNA if the same mature miRNA is generated from several separate loci (e.g. miR17a-1 and miR-17a-2). Two mature miRNAs can be produced by each locus, one from the 5′ strand and one from the 3′ strand (for example miR-17a-3p and miR17a-5p). Of note, one arm is usually more prevalent (named guide strand) than the other (named passenger strand, which is known as miRNA*) and more biologically active.

The RNA sequences of the miRs according to the invention are publicly available, for example on the mirbase (https://www.mirbase.org/index.shtml).

In particular, miR-17 covers both miR-17-3p, miR17-5p, miR-17-*, more particularly miR-17-3p, and it encompasses the following RNA sequences:

miR-17 of SEQ ID Nº 1:
GUCAGAAUAAUGUCAAAGUGCUUACAGUGCAGGUAGUGAUAUGUGCAUC
UACUGCAGUGAAGGCACUUGUAGCAUUAUGGUGAC;
miR17-3p of SEQ ID Nº 2:
ACUGCAGUGAAGGCACUUGUAG;
miR-17-5p of SEQ ID Nº 3:
CAAAGUGCUUACAGUGCAGGUAG.

In particular, miR-340 covers both miR-340-5p, miR340-3p, miR-340-*; more particularly miR-340-5 and it encompasses the following RNA sequences:

miR-340 of SEQ ID Nº 4:
UUGUACCUGGUGUGAUUAUAAAGCAAUGAGACUGAUUGUCAUAUGUCG
UUUGUGGGAUCCGUCUCAGUUACUUUAUAGCCAUACCUGGUAUCUUA;
miR-340-5p of SEQ ID Nº 5:
UUAUAAAGCAAUGAGACUGAUU;
miR-340-3p of SEQ ID Nº 6:
UCCGUCUCAGUUACUUUAUAGC.

In particular, miR-222 covers both miR-222-5p, miR222-3p, miR-222-*; more particularly it encompasses the following RNA sequences:

miR-222 of, SEQ ID Nº 7:
GCUGCUGGAAGGUGUAGGUACCCUCAAUGGCUCAGUAGCCAGUGUAGA
UCCUGUCUUUCGUAAUCAGCAGCUACAUCUGGCUACUGGGUCUCUGAU
GGCAUCUUCUAGCU;
miR-222-5p of SEQ ID Nº 8:
CUCAGUAGCCAGUGUAGAUCCU;
miR222-3p of SEQ ID Nº 9:
AGCUACAUCUGGCUACUGGGU.

In particular, miR-221 covers both miR-221-5p, miR221-3p, miR-221-*; more particularly it encompasses the following RNA sequences:

miR-221 of SEQ ID Nº 10:
UGAACAUCCAGGUCUGGGGCAUGAACCUGGCAUACAAUGUAGAUUUC
UGUGUUCGUUAGGCAACAGCUACAUUGUCUGCUGGGUUUCAGGCUAC
CUGGAAACAUGUUCUC;
miR-221-5p of SEQ ID Nº 11:
ACCUGGCAUACAAUGUAGAUUU;
miR-221-3p of SEQ ID Nº 12:
AGCUACAUUGUCUGCUGGGUUUC.

In particular, miR-551b covers both miR-551b-5p, miR551b-3p, miR-551b-*; more particularly it encompasses the following RNA sequences:

miR-551b of SEQ IDNº 13:
AGAUGUGCUCUCCUGGCCCAUGAAAUCAAGCGUGGGUGAGAC
CUGGUGCAGAACGGGAAGGCGACCCAUACUUGGUUUCAGAGG
CUGUGAGAAUAA;
miR-551b-5p of SEQ ID Nº 14:
GAAAUCAAGCGUGGGUGAGACC;
miR-551b-3p of SEQ ID Nº 15:
GCGACCCAUACUUGGUUUCAG.

As previously described, some overexpressed miRNAs have been reported to exert “oncogenic” effects while some downregulated miRNA showed “tumor suppressor” effects. Specific miRNA alterations can be specifically targeted by using oligonucleotide sequences referred to as “mimics” or “antagomirs” that induce an upregulation or downregulation of the targeted miRNAs, respectively. Therefore, by identifying the altered miRNAs in the tumor, the inventors have been able to put in place targeted therapy for personalized medicine in cancer patients, in particular for GBM patients.

The inventors have thus identified a combination of nucleic acids that act as mimics and optionally also as antogomiRs for the prevention and/or treatment of cancer.

By “mimic”, as used herein for miR-17mimic, miR-340 mimic and miR-551b mimic, it is referred to small, synthetic, double-stranded RNA molecules designed to mimic these miRNAs and as mentioned above, that are able to induce an upregulation of the targeted miRNAs. The mimic increases or replaces the intracellular function of said miRs and are capable of counteracting expression of the gene products the expression of which is downregulated by the miRNA which they are mimicking. miRNA mimic technology belongs to the miRNA-gain-of-function strategy, which is used to mimick an endogenous miRNAs so it can specifically bind to its targets, thereby inducing translational inhibition of the targeted gene.

Such molecules are known by the skilled person and are commercially available, and for example bought by the company Life technologies.

DNA-based miRNA mimics, RNA-based miRNA mimics, polynucleotides encoding a miRNA or a precursor thereof can be cited as examples.

DNA-based miRNA mimics correspond to DNA oligonucleotide comprising a DNA version of the sequence of the mature miRNA or a functionally variant equivalent thereof.

RNA-based miRNA mimics correspond to RNA-based mimics, which are double-stranded RNAs, wherein the sequence of one of the strands comprises the sequence of the mature miRNA or a functionally variant equivalent thereof.

In certain embodiments, the miRNA mimic is conjugated to one or more moieties which enhance the activity, cellular distribution or cellular uptake of the resulting antisense oligonucleotides. When the miRNA mimic is DNA-based or RNA-based comprising a single RNA polynucleotide stabilized by intrachain base pairing, the moiety may be connected to the 5′ end or to the 3′ end. When the miRNA mimic is RNA-based and double-stranded, the moiety may be connected to the 5′ end of the passenger strand, to the 3′ end of the guide strand, to the 5′ end of the guide strand and/or to the 3′ end of the guide strand.

As used herein, “guide strand” refers to a single-stranded nucleic acid molecule of an miRNA, which has a sequence sufficiently complementary to that of a target mRNA to hybridize to the target mRNA (e.g., in the 5′ UTR, the coding region, or the 3′ UTR) and to decrease or inhibit its translation. A passenger strand is also termed an “antisense strand.”

As used herein, “passenger strand” refers to an oligonucleotide strand of a miRNA, which has a sequence that is complementary or substantially complementary to that of the guide strand. In some embodiments, the passenger strand may target an mRNA by hybridizing to the target mRNA (e.g., in the 5′ UTR, the coding region, or the 3′ UTR) and to decrease or inhibit its translation A passenger strand is also termed a “sense strand.

In certain such embodiments, the moiety is a cholesterol moiety or a lipid moiety. Additional moieties for conjugation include carbohydrates, phospholipids, biotin, phenazine, folate, phenanthridine, anthraquinone, acridine, fluoresceins, rhodamines, coumarins, and dyes. In certain embodiments, a conjugate group is attached directly to an oligonucleotide. In certain embodiments, a conjugate group is attached to an oligonucleotide by a linking moiety selected from amino, hydroxyl, carboxylic acid, thiol, unsaturations (e.g., double or triple bonds), 8-amino-3,6-dioxaoctanoic acid (ADO), succinimidyl 4-(N-maleimidomethyl) cyclohexane-1-carboxylate (SMCC), 6-aminohexanoic acid (AHEX or AHA), substituted CI-CIO alkyl, substituted or unsubstituted C2-C10 alkenyl, and substituted or unsubstituted C2-C10 alkynyl. In certain such embodiments, a substituent group is selected from hydroxyl, amino, alkoxy, carboxy, benzyl, phenyl, nitro, thiol, thioalkoxy, halogen, alkyl, aryl, alkenyl and alkynyl.

In certain embodiments, the miRNA mimic is modified with a cap structure. This terminal modification protect an oligonucleotide from exonuclease degradation, and can help in delivery and/or localization within a cell. The cap can be present at the 5′-terminus (5′-cap), or at the 3′-terminus (3′-cap), or can be present on both termini. Cap structures include, for example, inverted deoxy abasic caps. Suitable cap structures include a 4′,5′-methylene nucleotide, a 1-(beta-D-erythrofuranosyl) nucleotide, a 4′-thio nucleotide, a carbocyclic nucleotide, a 1,5-anhydrohexitol nucleotide, an L-nucleotide, an alpha-nucleotide, a modified base nucleotide, a phosphorodithioate linkage, a threopentofuranosyl nucleotide, an acyclic 3′,4′-seco nucleotide, an acyclic 3,4-dihydroxybutyl nucleotide, an acyclic 3,5-dihydroxypentyl nucleotide, a 3′-3′-inverted nucleotide moiety, a 3′-3′-inverted abasic moiety, a 3′-T-inverted nucleotide moiety, a 3′-T-inverted abasic moiety, a 1,4-butanediol phosphate, a 3′-phosphoramidate, a hexylphosphate, an aminohexyl phosphate, a 3′-phosphate, a 3′-phosphorothioate, a phosphorodithioate, a bridging methylphosphonate moiety, and a non-bridging methylphosphonate moiety 5′-amino-alkyl phosphate, a 1,3-diamino-2-propyl phosphate, 3-aminopropyl phosphate, a 6-aminohexyl phosphate, a 1,2-aminododecyl phosphate, a hydroxypropyl phosphate, a 5′-5′-inverted nucleotide moiety, a 5′-5′-inverted abasic moiety, a 5′-phosphoramidate, a 5′-phosphorothioate, a 5′-amino, a bridging and/or non-bridging 5′-phosphoramidate, a phosphorothioate, and a 5′-mercapto moiety.

In a preferred embodiment, the mimics forming part of the composition according to the present invention mimic the human miRNAs. A human miRNA molecule is a miRNA molecule which is found in a human cell, tissue or organ. A human miRNA molecule may also be a human miRNA molecule derived from an endogenous human miRNA molecule by substitution, deletion and/or addition of a nucleotide.

By “antogomiR”, as used herein for miR-222 antagomiR and miR-221 antogomiR, it is referred to small, synthetic, single-stranded RNA molecules designed to specifically bind to and inhibit these miRNAs from functioning and, as mentioned above, that are able to induce a downregulation of the targeted miRNAs. AntagomiRs represent an antisense inhibition of miRNA approach. By binding to the targeted miRNA, they inhibit its function. Chemical modifications can be used to improve their stability, potency and performance.

Such molecules are known by the skilled person and are commercially available and for example bought by the company Life technologies.

Antagomirs can be chemically engineered cholesterol-conjugated single-strand RNA analogues that are efficient and specific silencers of endogenous microRNAs in vitro and in vivo. Inhibition of miRNAs can also be achieved with with antisense 2′-O-methyl (2′-OMe) oligoribonucleotides or by use of lentivirally or adenovirally expressed antagomirs. Furthermore, MOE (2′-O-methoxyethyl phosphorothioate) or LNA (locked nucleic acid (LNA) phosphorothioate chemistry)-modification of single-stranded RNA analogous can be used to inhibit miRNA activity.

In addition, endogenous miRNAs can be silenced by the use of miRNA sponges. In that case a single species of RNA is constructed, that contains multiple, tandem-binding sites for a miRNA seed family of interest. As various members of a miRNA seed family are targeted, the potential advantage of this approach is to more effectively influence disease pathways commonly regulated by this family of miRNAs. In principle it is possible to interfere with miRNA function by scavenging away the miRNA and thereby preventing it from binding its mRNA targets.

The binding of a miRNA with a specific mRNA target can also be prevented using an oligonucleotide with perfect complementary to the miRNA target sequence in the 3′-UTR of the mRNA, which thereby masks the binding site and prevents association with the miRNA. A further approach to inhibit miRNA function can be achieved by “erasers,” in which expression of a tandem repeat of a sequence perfectly complementary to the target miRNA inhibits the endogenous miRNA function. Finally substances, molecules, drugs can be used to inhibit miRNA expression and biogenesis.

By delivering single-stranded oligonucleotides equivalent of the mature miRNA, an increase in the effective concentration of a reduced miRNA can be achieved through the use of synthetic RNA duplexes in which 1 strand is identical to the native miRNA. In this case, short double-stranded oligonucleotides are designed in which 1 strand is the mature miRNA sequence (guide strand) and a complimentary or partially complementary stand is complexed with the mature miRNA sequence (passenger strand). Finally substances, molecules, drugs can be used to increase miRNA expression and biogenesis.

In a preferred embodiment, the antagomiRs forming part of the composition according to the present invention target the human miRNAs.

The terms “treat”, “treating”, “treated” or “treatment”, as used in the context of the invention, refer to therapeutic treatment wherein the object is to eliminate or lessen symptoms. Beneficial or desired clinical results include, but are not limited to, elimination of symptoms, alleviation of symptoms, diminishment of extent of condition, stabilized (i.e., not worsening) state of condition, delay or slowing of progression of the condition.

The terms “prevent”, “prevention”, “preventing” or “prevented”, as used in the context of the present invention, refer to the prevention of the onset, recurrence or spread of a disease or disorder, or of one or more symptoms thereof. In certain embodiments, the terms refer to the treatment with or administration of a compound provided herein prior to the onset of symptoms, particularly to patients at risk of disease or disorders provided herein. The terms encompass the inhibition or reduction of a symptom of the particular disease. Subjects with familial history of a disease in particular are candidates for preventive regimens in certain embodiments. In addition, subjects who have a history of recurring symptoms are also potential candidates for the prevention. In this regard, the term “prevention” may be interchangeably used with the term “prophylactic treatment”.

As used herein and unless otherwise defined, “cancer” refers to the growth, division or proliferation of abnormal cells in the body. It refers to any type of malignant (i.e. non benign) tumor. The malignant tumor may correspond to a primary tumor or to a secondary tumor (i.e. a metastasis). Further, the tumor may correspond cancer chosen from brain tumors; medulloblastomas; retinoblastomas; schwannomas; neuroblastomas; melanomas; NSCLCC; pancreatic tumors; breast cancers, hepatocarcinomas and nephroblastomas.

In particular, the present invention relates to a combination as herein described for use for the prevention and/or treatment of brain tumors; medulloblastomas; retinoblastomas; schwannomas; neuroblastomas; melanomas; NSCLCC; pancreatic tumors; breast cancers, hepatocarcinomas and nephroblastomas.

More particularly, the brain tumor is chosen from astrocytomas, oligodendrogliomas, meningiomas, gliomas and glioblastomas, and even more particularly the cancer to prevent and/or treat is glioblastoma.

As already mentioned, glioblastoma (GBM) is a grade IV astrocytoma, a deadly malignant brain tumor and among the most common primary brain tumor in adults.

In particular, GBM can be characterized into different subtypes: mesenchymal, proneural, neural, and classical.

Accordingly, in one embodiment, the combination according to the invention can be used for the prevention and/or treatment of glioblastomas of one or more of these subtypes.

Most importantly, in one embodiment, the combination according to the invention is effective to prevent or treat all these subtypes.

The present invention also relates to a method of prevention and/or treatment of a cancer, in particular GBM, comprising the administration to a subject in need thereof of an effective amount of a combination as described herein.

In particular, the subject in need of a treatment against cancer is a subject afflicted with such disease.

In the context of the present invention, the identification of the subjects who are in need of treatment of herein-described diseases and conditions is conducted as above mentioned and is well within the ability and knowledge of the man skilled in the art. A clinician skilled in the art can readily identify, by the above mentioned technics, those subjects who are in need of such treatment.

A therapeutically effective amount can be readily determined by the attending diagnostician, as one skilled in the art, by the use of conventional techniques and by observing results obtained under analogous circumstances. In determining the therapeutically effective amount, a number of factors are considered by the attending diagnostician, including, but not limited to: the species of subject; its size, age, and general health; the specific disease involved; the degree of involvement or the severity of the disease; the response of the individual subject; the particular compound administered; the mode of administration; the bioavailability characteristic of the preparation administered; the dose regimen selected; the use of concomitant medication; and other relevant circumstances.

As used herein, an “effective amount” refers to an amount, which is effective in reducing, eliminating, treating or controlling the symptoms of the herein-described diseases and conditions. The term “controlling” is intended to refer to all processes wherein there may be a slowing, interrupting, arresting, or stopping of the progression of the diseases and conditions described herein, but does not necessarily indicate a total elimination of all disease and condition symptoms, and is intended to include prophylactic treatment and chronic use.

The term “patient” or “subject” refers to a warm-blooded animal such as a mammal, in particular a human, male or female, unless otherwise specified, which is afflicted with, or has the potential to be afflicted with one or more diseases and conditions described herein.

The amount of each mimic or antagomiR, which is required to achieve the desired biological effect, will vary depending upon a number of factors, including the dosage of the drug to be administered, the chemical characteristics (e.g. hydrophobicity) of the compounds employed, the potency of the compounds, the type of disease, the diseased state of the patient, and the route of administration.

Combinations provided herein can be formulated into pharmaceutical compositions, optionally by admixture with one or more pharmaceutically acceptable excipients.

Such compositions may be prepared for use in oral administration, particularly in the form of tablets or capsules, in particular orodispersible (lyoc) tablets; or parenteral administration, particularly in the form of liquid solutions, suspensions or emulsions.

It may be prepared by any of the methods well known in the pharmaceutical art, for example, as described in Remington: The Science and Practice of Pharmacy, 20^{th} ed.; Gennaro, A. R., Ed.; Lippincott Williams & Wilkins: Philadelphia, PA, 2000. Pharmaceutically compatible binding agents and/or adjuvant materials can be included as part of the composition. Oral compositions will generally include an inert diluent carrier or an edible carrier. They can be administered in unit dose forms, wherein the term “unit dose” means a single dose which is capable of being administered to a patient, and which can be readily handled and packaged, remaining as a physically and chemically stable unit dose comprising either the active compound itself, or as a pharmaceutically acceptable composition.

The tablets, pills, powders, capsules, troches and the like can contain one or more of any of the following ingredients, or compounds of a similar nature: a binder such as microcrystalline cellulose, or gum tragacanth; a diluent such as starch or lactose; a disintegrant such as starch and cellulose derivatives; a lubricant such as magnesium stearate; a glidant such as colloidal silicon dioxide; a sweetening agent such as sucrose or saccharin; or a flavoring agent such as peppermint, or methyl salicylate. Capsules can be in the form of a hard capsule or soft capsule, which are generally made from gelatin blends optionally blended with plasticizers, as well as a starch capsule. In addition, dosage unit forms can contain various other materials that modify the physical form of the dosage unit, for example, coatings of sugar, shellac, or enteric agents. Other oral dosage forms syrup or elixir may contain sweetening agents, preservatives, dyes, colorings, and flavorings. In addition, the active compounds may be incorporated into fast dissolve, modified-release or sustained-release preparations and formulations, and wherein such sustained-release formulations are preferably bi-modal.

Liquid preparations for administration include sterile aqueous or non-aqueous solutions, suspensions, and emulsions. The liquid compositions may also include binders, buffers, preservatives, chelating agents, sweetening, flavoring and coloring agents, and the like. Non-aqueous solvents include alcohols, propylene glycol, polyethylene glycol, acrylate copolymers, vegetable oils such as olive oil, and organic esters such as ethyl oleate. Aqueous carriers include mixtures of alcohols and water, hydrogels, buffered media, and saline. In particular, biocompatible, biodegradable lactide polymer, lactide/glycolide copolymer, or polyoxyethylene-polyoxypropylene copolymers may be useful excipients to control the release of the active compounds. Intravenous vehicles can include fluid and nutrient replenishers, electrolyte replenishers, such as those based on Ringer's dextrose, and the like.

Examples of modes of administration include parenteral e.g. subcutaneous, intramuscular, intravenous, intradermal, as well as oral administration, in particular oral, intravenous, or subcutaneous administration.

The present invention will be further illustrated by the following figures and examples.

### FIGURES

FIG. 1: MiR-17-3p, -340, and -222 modulate GBM cell survival, clonogenicity and transmigration.
(A). miRNA expression was determined by qPCR in Ge518 transfected by non-targeting scrambled control or mimics of miR-17-3p, -340, -551b, and -222 antagomir (n=5-6).
(B). Cell viability of Ge518, Ge738, Ge904 and Ge970.2 transiently transfected with non-targeting scrambled control or mimics of miR-340, -17-3p, 551b and -222 antagomir was evaluated after three or four days using CellTiter-Glo. Histograms represent the fold change of cell survival for the miR-Combo versus the miR-Ctrl (n=4-5).
(C). Clonogenicity of Ge518, Ge738, Ge904 and Ge970.2 transiently transfected with non-targeting scrambled control or mimics of miR-340, -17-3p, and -222 antagomir was determined using the clonogenic assay. Representative pictures of 3-4 independent experiments. Histograms represent the fold change of clones formed in for miR-Combo versus the miR-Ctrl condition. Scale bar=10 μm.
(D). Transmigration of Ge518, Ge738, Ge904 and Ge970.2 transiently transfected with non-targeting scrambled control or mimics of miR-340, -17-3p, and -222 antagomir was determined using transwell plates. Representative pictures of 3 independent experiments. Histograms represent the fold change of transmigrated cells through the transwell for the miR-Combo versus the miR-Ctrl condition. Scale bar=10 μm.
Data are represented as mean±SEM (*p<0.05, **p<0.01 and ***p<0.001), ns=non-significant.

FIG. 2: (A-E). miRNA expression was determined by qPCR in Ge518 transfected by non-targeting scrambled (A) control or mimics of (B) miR-17-3p, (C) -340, (D) -551b, and (E) -222 antagomir (n=5-6). Histograms show the expression of each miRNA in each condition normalized to the housekeeping genes. (F-1). Cell viability of Ge518, Ge738, Ge904 and Ge970.2 transiently transfected with non-targeting scrambled control or mimics of miR-17-3p (F), -340 (G), 551b (H) and -222 (1) antagomir was evaluated after three or four days using CellTiter-Glo. Histograms represent the fold change of cell survival for the miR-Combo versus the miR-Ctrl (n=4-5). Data are represented as mean±SEM (*p<0.05, **p<0.01 and ***p<0.001), ns=non-significant.

FIG. 3: (A-D). Clonogenicity of Ge518, Ge738, Ge904 and Ge970.2 transiently transfected with non-targeting scrambled control or mimics of miR-17-3p (A), -340 (B), -551b (C) and -222 (D) antagomir was determined using the clonogenic assay. Histograms represent the fold change of clones formed in each condition versus the miR-Ctrl condition. (E). Representative pictures of the 3-4 independent experiments. Scale bar=1 μm. Data are represented as mean±SEM (*p<0.05, **p<0.01 and ***p<0.001), ns=non-significant.

FIG. 4: Transmigration of Ge518, Ge738, Ge904 and Ge970.2 transiently transfected with non-targeting scrambled control or mimics of miR-17-3p (A), -340 (B), -551b (C) and -222 (D) antagomir was determined using transwell. Histograms represent the fold change of transmigrated cells through the transwell for each condition versus the miR-Ctrl. Representative pictures of the 3 independent experiments. Scale bar=1 μm.
Data are represented as mean±SEM (*p<0.05, **p<0.01 and ***p<0.001), ns=non-significant.

FIG. 5: The combinatorial modulation of miR-340, -17-3p and -222 regulates genes involved in several biological processes and metabolic pathways.
(A). Functional annotation clustering of gene set enrichment analysis showing Ge518 and Ge970.2 transfected with a non-targeting scrambled control or the combinatorial modulation of miR-340, -17-3p and -222. Histograms show the number of query enriched for each family of genes from g: Profiler analysis.
(B). Venn diagram of comparisons between Ge518 and Ge970.2 transfected with a non-targeting scrambled control or the combinatorial modulation of miR-340, -17-3p and -222.
(C). Hierarchical clustering of Ge518 and Ge970.2 miR-Ctrl vs. miR-Combo based on the differential expressed genes.
(D). Functional annotation clustering of gene set enrichment analysis comparing Ge518 and Ge970.2 transfected with a non-targeting scrambled control or the combinatorial modulation of miR-340, -17-3p and -222. Histograms show the number of queries enriched for each family of genes from g: Profiler analysis.
Data are represented as mean±SEM (*p<0.05, **p<0.01 and ***p<0.001).

FIG. 6: The modulation of miR-340, -17-3p and -222 regulate gene involved in several signaling pathways. (A). Functional annotation clustering of gene set enrichment analysis comparing Ge518 transfected with a non-targeting scrambled control or mimics of miR-17-3p, -340, and -222-3/5p antagomir. Histograms show the enrichment index of each family of genes. (B-D). Hierarchical clustering of Ge518 transiently transfected with non-targeting scrambled control or mimics of miR-340 (B), -17-3p (C), and -222-3/5p antagomir (D) for 24 h based on the differential expressed genes. (E). mRNA was determined by qPCR in Ge518 transfected by non-targeting scrambled control or mimics of miR-17-3p, -340, and -222-3/5p antagomir (n=3). (F). protein was determined by western blot in Ge518 transfected by non-targeting scrambled control or mimics of miR-17-3p, -340, and -222-3/5p antagomir (n=5). Data are represented as mean±SEM (*p<0.05, **p<0.01 and ***p<0.001), ns=non-significant.

FIG. 7: (A). Venn diagram of comparisons between miRNA potential target genes identified by RNASeq or with TargetScan. Ge518 and Ge970.2 transfected with a non-targeting scrambled control or the combinatorial modulation of miR-340, -17-3p and -222. (B). Functional annotation clustering of gene set enrichment analysis comparing the differential expressed genes identified in the RNASeq of miR-Combo versus miR-Ctrl and the RNASeq mimics of miR-17-3p, -340, and -222 antagomir. Histograms show the enrichment index of each family of genes. (C). Ge904 PDCs were co-transfected with 3′UTR reporter constructs and either miR-17-3p or miR-340 and luciferase activity was normalized to the respective control.

FIG. 8: The combinatorial modulation of miR-340, -17-3p and -222 induced an activation of AKT in GBM PDCs.
(A). The human phosphor-kinase array showed the relative expression of phosphorylation profiles of several kinases and their protein substrates.
Representative pictures of 3 independent experiments. Histograms represented the fold change of each kinases for the miR-Combo versus the miR-Ctrl condition.
(B). The human phospho-kinase array showed the relative expression of phosphorylation profiles of several kinases and their protein substrates. Representative pictures of 3 independent experiments.
(C). Immunoblots show expression of indicated proteins for Ge518 transfected with a non-targeting scrambled control or the combinatorial modulation of miR-340, -17-3p and -222. Histograms showed the fold change of protein expression determined by densitometry analysis.

FIG. 9: GBM PDC invasion in neural organoid was reduced by the combinatorial modulation of miR-17-3p, miR-222, and miR-340.
(A-D). Schematic representation of PSC differentiation towards neural organoids. PSC were cultured on Matrigel (A) then aggregated in microwell plates (B) for three weeks (C). (D) Schematic representation of the culture principle for neural organoids.
(E). Immunofluorescence showed NeuN, βII-Tubulin, GFAP, and MAP2-immunoreactive cells present in the neural organoids. Scale bar=100 μm.
(F). GAD67, Musashi, Nestin (NES), OLIG2, PSD95, S100B, SOX2 and βIII-Tubulin (TUBB3) mRNA expression was determined by qPCR in the neural organoids before co-culturing with GSC Ge904. Data were normalized to housekeeping genes; mean (n=4)±SEM.
(G). Immunofluorescence showed GFAP, and βIII-Tubulin-immunoreactive cells present in the co-culture of GSC Ge904 and neural organoid. Scale bar=100 μm. Histograms represented the invasive score of the GBM cells within the neural organoid.
(H). Immunofluorescence shows Ki-67, and βIII-Tubulin-immunoreactive cells presented in the neural organoids. Scale bar=100 μm. Histograms represented the proliferative score of the GBM cells within the neural organoid.
Data are represented as mean±SEM (*p<0.05, and **p<0.01).

FIG. 10: Repeated injection of miR-Combo delay tumor growth in nude mice.
(A). Effect of the combinatorial modulation of miR-17-3p, miR-222, and miR-340 on Ge518 tumor growth in vivo (n=5 mice per group).
(B). Histological analysis of Ge518 tumor treated with miR-Combo. Tumors were stained for the identified proteins, and counterstained with haematoxylin. Scale bar, 50 μm.
(C). Histograms represent the fold change of protein expression quantified by using ImageJ (n=3). Data are represented as mean±SEM (**p<0.01), ns=non significant.

FIG. 11: GSC bearing a stable doxycycline-inducible lentivector system expressing miR-17-3p, miR-222, and miR-340 induced a decrease of cell viability and delay tumor growth in vivo.
(A). Cell viability of Ge518, Ge738, and Ge970.2 PDCs expressing miRGE was evaluated after three days using CellTiter-Glo. Histograms represent the fold change of cell survival for the miRGE treated with doxycycline (Dox) versus untreated.
(B). Cell viability of Ge518, Ge738, and Ge970.2 GSCs expressing miRGE was evaluated after three days using CellTiter-Glo. Histograms represent the fold change of cell survival for the miRGE treated with Dox versus untreated.
(C). Representative pictures of 3-7 independent experiments. The bar graph represents the fold change of GSC miRGE treated with Dox versus untreated. Data are represented as mean±SEM (*p<0.05, **p<0.01 and ***p<0.001).
(D). Effect of miRGE expression turned on by Dox treatment on tumor growth in vivo: a mixture of Ge518, Ge738, and Ge970.2 GSCs (1:1:1) bearing the inducible Tet-On system was intracranially injected into the brain of nude mice (n=8 mice in the treated group; n=7 mice in the untreated group). Log-rank Mantel-Cox test was used to calculate significance.

FIG. 12: Cell viability of A375 (melanoma cell line), MCF-7 (breast cell line), A549 (lung cancer cell line) and Ge360 (ependymoma cell line) transiently transfected with non-targeting scrambled control or mimics of miR-340, -17-3p, and -222 antagomir was evaluated after three or four days using CellTiter-Glo. Histograms represent the fold change of cell survival for the miR-Combo versus the miR-Ctrl (n=3).

### EXAMPLES

### Example 1

### Material and Methods

### GBM Cell Lines and Patient-Derived Models

Eight Glioblastoma Stem Cells (GSCs) from different subtypes Ge269, 518, 835, (Mesenchymal subtype) 738, 898, (proneural subtype) 885, (Neural subtype) 904, 970.2 (classical subtype) were cultured in DMEM/F12 with Glutamax supplemented with B27 supplement and b-FGF, EGF both at 10 ng/ml, 1% penicillin/streptomycin, as previously described (Cosset, E, et al. (2016). Human tissue engineering allows the identification of active miRNA regulators of glioblastoma aggressiveness. Biomaterials 107: 74-87). To generate GBM patient-derived cell (PDCs), we transferred the GSCs to, and maintained them in, Dulbecco's modified Eagle's medium (DMEM)-high glucose/glutamax, 10% Fetal Bovine Serum (FBS), 1% penicillin/streptomycin (GDC medium).

### Chemicals

Actinomycin D and Doxycycline, were obtained by Sigma and used at the concentration of 5 μM and 1 mg/ml for 24 hours, respectively.

### Cell Transfection

The miR-17-3p, miR-340-5p and miR-551b mimics, and miR-222-3p antagomir were transfected using lipofectamine RNAimax (Invitrogen), at a final concentration of 5 nM, according to the manufacturer's protocols. A non-targeting scrambled miRNA (Life Technologies) was used as control.

### Cell Viability Assay

Cell viability assay was performed by using CellTiter-Glo assay kit (Promega) as previously described (Cosset, E, et al. (2016). Human tissue engineering allows the identification of active miRNA regulators of glioblastoma aggressiveness. Biomaterials 107: 74-87; Cosset, E, et al. (2017). Glut3 Addiction Is a Druggable Vulnerability for a Molecularly Defined Subpopulation of Glioblastoma. Cancer Cell 32: 856-868 e855).

### Cell Transmigration

Cells (2×104) were seeded onto the filters of transwell polycarbonate membrane inserts (24 well, 8 μm pores, Corning) in serum free (0% FBS) GDC medium and the lower compartments were filled with 10% FBS GDC medium. After 24 hours of incubation at 37° C. to allow transmigration assessment, the adherent cells on the lower surface were stained with 0.1% crystal violet and quantified and/or counted. Images were captured using EVOS microscope (Life Technologies) and manually counted.

mIRGE Lentivector

miRGE construct bearing miR-17-3p, miR-340-5p and miR-222-3p was generated by Vector Lab (Dr. Patrick Salmon, University of Geneva), as previously reported (Myburgh, R, et al. (2014). Optimization of Critical Hairpin Features Allows miRNA-based Gene Knockdown Upon Single-copy Transduction. Mol Ther Nucleic Acids 3: e207).

### Luciferase Assay

At day 0 (DO), PDCs Ge904, chosen because they don't express miR-340 and miR-17-3p, were seeded at 80,000 cells per well in a 24 well plate. 24 h later at D1, cells were transfected with the corresponding miRNA and plasmid using Lipofectamine 3000 according to the manufacturer's protocol (L3000-008 Invitrogen). 24 h later at D2, cells were lysed in the wells using the passive Lysis buffer from the Dual Luciferase Reporter Assay (E1910 Promega), followed by a measurement of the luminescence according to the manufacturer's protocol. Results were normalized to Renilla control.

### Neural Organoids

Human induced pluripotent stem cell line (iPSCs) and embryonic stem cells (ESCs) were used to generate the neural organoids, as previously described with minor modifications (Cosset, E, et al. (2019). Human Neural Organoids for Studying Brain Cancer and Neurodegenerative Diseases. J Vis Exp.). iPSCs was kindly provided by Dr. Youssef Hibaoui and generated as previously described (Hibaoui, Y, et al. (2014). Modelling and rescuing neurodevelopmental defect of Down syndrome using induced pluripotent stem cells from monozygotic twins discordant for trisomy 21. EMBO Mol Med 6: 259-277.). The human ESCs, HS420, was kindly provided by the Professor Karl-Heinz Krause.

### Human Phospho-Kinase Assay

The phosphorylation profiles of kinases were performed using the human phospho-kinase array kit (R&D Systems) according to the manufacturer's recommendations.

For quantification, dots were analyzed on Image J software using the analyze gels, plot lane command. All dots were normalized to the negative control. Combo conditions were normalized to the control conditions.

### Immunoblotting

Proteins were extracted in IP-MS cell lysis buffer (Life Technologies) and quantified using the Pierce BCA kit (Thermo Fisher) as previously described (Cosset, E, et al. (2017). Glut3 Addiction Is a Druggable Vulnerability for a Molecularly Defined Subpopulation of Glioblastoma. Cancer Cell 32: 856-868 e855). The following antibodies were used for immunoblotting: Vimentin (Millipore), p-P70 S6 Kinase (Cell Signaling), P70 S6 Kinase (Cell Signaling), pAKT (Cell Signaling), AKT (Cell Signaling), GAPDH (Cell Signaling), and β-actin HRP (Sigma-Aldrich) as loading control. For protein expression analysis, expression was normalized to β-actin then compared to their respective control.

Immunohistochemistry (IHC) and immunofluorescence (IF) IHC and IF staining of formalin-fixed paraffin-embedded tissues was carried out as previously described (Cosset, E, et al. (2016). Human tissue engineering allows the identification of active miRNA regulators of glioblastoma aggressiveness. Biomaterials 107: 74-87; Cosset, E, et al. (2017). Glut3 Addiction Is a Druggable Vulnerability for a Molecularly Defined Subpopulation of Glioblastoma. Cancer Cell 32: 856-868 e855). Sections were then incubated overnight at 4° C. with primary antibody pAKT (Cell Signaling), AKT (Cell Signaling), Ki-67 (Chemicon), and CD31 (Abcam) followed by, for IHC, biotin-conjugated anti-rabbit IgG and an avidin-biotin peroxidase detection system with 3,3′-diaminobenzidine substrate (Vector), then counterstained with hematoxylin (Sigma). For IF, sections were then incubated overnight at 4° C. with primary antibody βIII-Tubulin (Covance), GFAP (Dako), MAP-2 (Millipore), NeuN (Millipore), Ki-67 (Chemicon). Then, fluorochrome-labeled secondary antibodies were used: Alexa Fluor (555 and/or 488)-labeled antibodies from goat or donkey against mouse, goat, or rabbit (Molecular Probes).

### Reverse Transcription Quantitative PCR (RT-qPCR)

Isolation of total RNA was carried out by using RNeasy kit from Qiagen according to the manufacturer's instructions and as previously described (Cosset, E, et al. (2016). Human tissue engineering allows the identification of active miRNA regulators of glioblastoma aggressiveness. Biomaterials 107: 74-87). Primer sequences are described in Tables 1 and 2.

TABLES 1 and 2

| Gene | Forward | Reverse |
|---|---|---|
| VIM | GACGCCATCAACACCGAGTT | CTTTGTCGTTGGTTAGCTGGT |
| | (SEQ ID Nº 16) | (SEQ ID Nº 17) |
| RAP2A | CGTGAAGCGGTATGAGAAAGT | GCCTTCGCTGGACGATACT |
| | (SEQ ID Nº 18) | (SEQ ID Nº 19) |
| LITAF | ATGTCGGTTCCAGGACCTTAC | TACGAAGGAGGATTCATGCCC |
| | (SEQ ID Nº 20) | (SEQ ID N°21) |
| NFKBIZ | ACACCCACAAACCAACTCTGG | GGAAAACTGTGATTCTGGACC |
| | (SEQ ID N°22) | (SEQ ID Nº 23) |
| ROCK1 | AACCATGTGACTGAGTGCCC | TCAGTGTGTTGTGCCAAAGC |
| | (SEQ ID Nº 24) | (SEQ ID Nº 25) |
| CHRNA4 | GGAGGGCGTCCAGTACATTG | GAAGATGCGGTCGATGACCA |
| | (SEQ ID Nº 26) | (SEQ ID Nº 27) |
| BDKRB2 | GTCTGTTCGTGAGGACTCCG | AAAGGTCCCGTTAAGAGTGGG |
| | (SEQ ID Nº 28) | (SEQ ID Nº 29) |
| GALNT3 | TCTTGGACCAGACACTCGAC | GGACAGTTCTAAGCAACGTCC |
| | (SEQ ID N°30) | (SEQ ID Nº 31) |
| STMN3 | CGCACCCCAATACCGTCTAC | GGACAGGTCAGAAGGGGACTT |
| | (SEQ ID Nº 32) | (SEQ ID Nº 33) |
| COL5A3 | GTGGCCGTCAGCATAGATGG | TGAATGTCTCCCTCGAAAGTCTT |
| | (SEQ ID Nº 34) | (SEQ ID Nº 35) |
| EEF1A1 | AGCAAAAATGACCCACCAATG | GGCCTGGATGGTTCAGGATA |
| | (SEQ ID N°36) | (SEQ ID Nº 37) |
| ALAS1 | CTCACCACACACCCAGATG | AGTTCCAGCCCACTTGCT |
| | (SEQ ID Nº 38) | (SEQ ID Nº 39) |

| miRNAs | Sequence |
|---|---|
| miR-340-5p | UUAUAAAGCAAUGAGACUGAUU |
| | (SEQ ID Nº 5) |
| miR-17-3p | ACUGCAGUGAAGGCACUUGUAG |
| | (SEQ ID Nº 2) |
| miR-222-3p | AGCUACAUCUGGUACUGGGU |
| | (SEQ ID Nº 9) |
| miR-222-5p | CUCAGUAGCCAGUGUAGAUCCU |
| | (SEQ ID Nº 8) |
| miR-551b-5p | GAAAUCAAGCGUGGGUGAGACC |
| | (SEQ ID Nº 14) |
| miR-16-5p | UAGCAGCACGUAAAUAUUGGCG |
| | (SEQ ID Nº 40) |
| miR-191-5p | CAACGGAAUCCCAAAACAGCUG |
| | (SEQ ID Nº 41) |

Prior to utilization, an efficacy test was performed, and all primers were validated. At least, two housekeeping genes (EEF1A1, and ALAS1) were used for normalization. RT-PCR reactions were performed in, at least, three technical and biological triplicates, and the average cycle threshold (CT) values were determined. For miRNAs, miR-16-5p and miR-191-5p were validated and used as housekeeping genes.

### Analysis of RNASeq Data

As previously described (Cosset, E, et al. (2016). Human tissue engineering allows the identification of active miRNA regulators of glioblastoma aggressiveness. Biomaterials 107: 74-87), total RNA was extracted using the Trizol method. Then, after checking RNA quality, the SR100—libraries TruSeqHT stranded—Illumina HiSeq 4000 was used and the sequencing quality control was done with FastQC v.0.11.5.

Then, hierarchical clustering were generated through Morpheus (https://software.broadinstitute.org/morpheus).

### In Silico Data Analysis

Gliovis was used to retrieve p-value for Kaplan-Meier log-rank-test analysis of target genes from the TCGA dataset (http://gliovis.bioinfo.cnio.es/). The gene enrichment analysis was done using g:Profiler and DAVID Bioinformatics resources (Raudvere, U, et al. (2019). g:Profiler: a web server for functional enrichment analysis and conversions of gene lists (2019 update). Nucleic Acids Res 47: W191-W198; Huang da, W, et al. (2009). Bioinformatics enrichment tools: paths toward the comprehensive functional analysis of large gene lists. Nucleic Acids Res 37: 1-13.; Huang da, et al. (2009). Systematic and integrative analysis of large gene lists using DAVID bioinformatics resources. Nat Protoc 4: 44-57).

### TCGA Analysis

miRNA expression data and the corresponding clinical data for GBM samples were downloaded (http://cancergenome.nih.gov/) from the TCGA data portal and analyzed as previously described (Cosset, E, et al. (2016). Human tissue engineering allows the identification of active miRNA regulators of glioblastoma aggressiveness. Biomaterials 107: 74-87).

### Subcutaneous Injection

All animal procedures were performed in accordance with ethics committee for animal research of Geneva under the approved protocol GE/38/20 according to the standards set by the institutional animal care. Ge518, and Ge738 GSCs (5×106 tumor cells in 200 μl of PBS) were injected subcutaneously into the right flank of mice. Tumor sizes were monitored three times per week with caliper until they reached a size of 150 mm3.

### Dosing

The mimic-invivofectamine and antagomir-invivofectamine complex was prepared according to the manufacturer's instructions. The final concentration of mimic/antagomir was 1.75 mg/ml per 200 μl per mouse (corresponding to 5 nmol). As a first pulse, mice were injected twice with the aforementioned concentration, then every other day with 0.875 mg/ml per 200 μl per mouse (corresponding to 2.5 nmol). The solution was maintained at room temperature until injection. The mirVana™ negative control mimic was used for the control group. For the mimics, we used mirVana™ miR-17-3p (MC12246), mirVana™ miR-340-5p (MC12670) and for the antagomir mirVana™ miR-222 (MH11376).

### Orthotopic Brain Tumor Injection

Ge518, Ge738, and Ge970.2 bearing miRGE were orthotopically transplanted following washing and resuspension in PBS into 6-10-week-old female nu/nu immunocompromised mice.

### Quantification and Statistical Analysis

Sample size and statistics for each experiment are provided in the Results section and Figure Legends. Data shown are representative of results obtained for multiple experiments as noted in the Figure Legends. All statistical analyses were performed using one-way analysis of variance (ANOVA) and Student's t-test, with p<0.05 considered significant. All statistical analyses were carried out using Prism software GraphPad). Chi-squared tests or t-tests were used to calculate statistical significance.

### Results

The Combinatorial Modulation of miR-17-3p, miR-222, and miR-340 Inhibits GBM Cell Viability, Clonogenicity and Transmigration Capacity

We first selected PDCs where miR-17-3p, miR-340 and miR-551b were not expressed or expressed at a low level, and where miR-222 was expressed. Indeed, we aimed at increasing miR-17-3p, miR-340 and miR-551b expression and decreasing miR-222 expression. After screening nine patient-derived GBM cells and two established cell lines (U87MG and U251) by using quantitative RT-PCR (RT-qPCR), our results revealed that Ge518 was the best patient-derived cell (PDC) model for modulating miRNA expression (Table 3).

TABLE 3
miRNA expression evaluated by qPCR in all GBM cell lines. Data are represented as mean
of 3 independent experiments. Grey rows represent no to low expression; + = fold change

| | mir340-3p | mir340-5p | mir551b-5p | mir551-3p | mir17-3p | mir17-5p | mir222-3p | mir222-5p |
|---|---|---|---|---|---|---|---|---|
| 269 | ++ | ++ | | | | + | | |
| 518 | | | | | + | + | | +++ |
| 688 | | + | | | | + | | + |
| 738 | + | + | | | +++ | +++ | | +++ |
| 885 | + | | | | | | | |
| 898 | + | + | | | | | | |
| 970.2 | + | | | | | | | |
| 835 | | | | | | | ++ | |
| 904 | | | | + | | | | |
| U87 | | | | | | + | + | |
| U251 | | | | | | | + | |

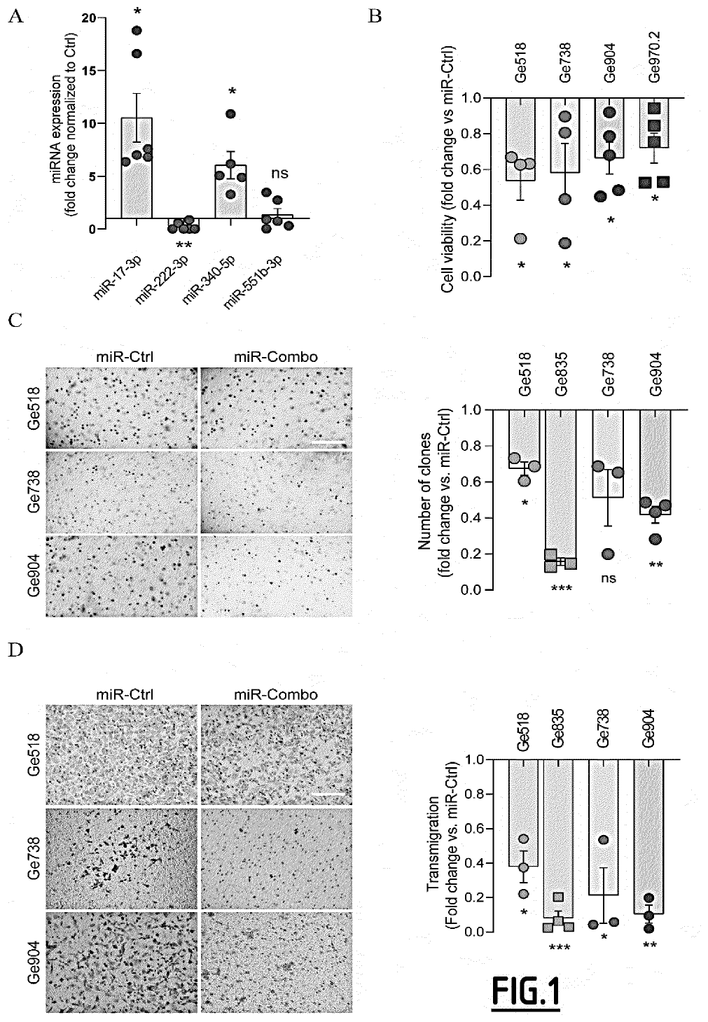

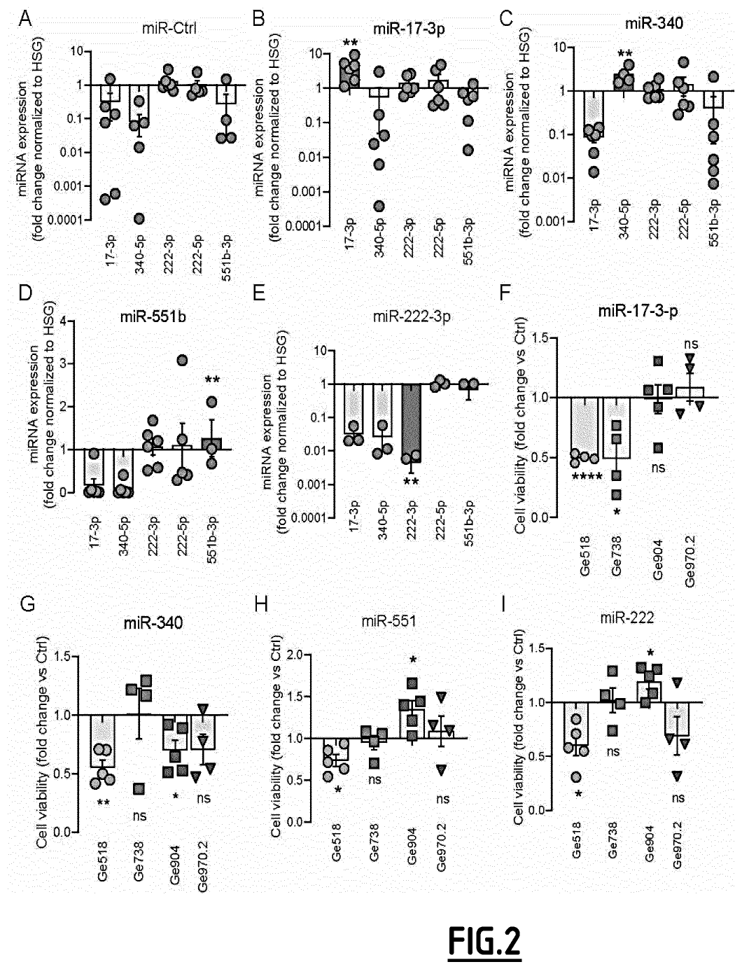

We reasoned that a miRNA-based multi-targeting therapy would be more beneficial for GBM patients. To test this hypothesis, combined mimics and antagomir (miR-Combo) were used to either ectopically express or inhibit expression, respectively, in Ge518 PDCs. After confirming miRNA modulation by quantitative stem-loop real-time RT-PCR (FIG. 1A), we measured cell viability at three days post-transfection (FIG. 1B) and observed a significant decrease of cell viability with miR-Combo compared to the non-targeting scrambled control (miR-Ctrl). As previously reported, Ge518 showed a mesenchymal phenotype (Cosset, et al. (2017). Glut3 Addiction Is a Druggable Vulnerability for a Molecularly Defined Subpopulation of Glioblastoma. Cancer Cell 32: 856-868.e855). To confirm the inhibitory potential of the miR-Combo on other patient-derived models with proneural and classical phenotypes, we also tested Ge738 (proneural subtype), Ge904 and Ge970.2 (classical subtype) (Cosset, et al. (2017). Glut3 Addiction Is a Druggable Vulnerability for a Molecularly Defined Subpopulation of Glioblastoma. Cancer Cell 32: 856-868.e855) cell viability after transfection (FIG. 2B). Our results revealed a significant inhibition of cell viability for all PDCs, indicating that GBM cells from any subtype with differential expression of the identified miRNAs can be therapeutically targeted with the miR-Combo. Then, we sought to understand which miRNA was responsible for this inhibition. Consequently, we transfected each miRNA separately, confirmed their modulation (Supplementary FIG. 2A-D), and analyzed PDC viability. As shown in the Supplementary FIG. 2F-1, we observed a significant decrease of cell viability of several PDCs mainly through miR-17-3p and miR-340. A trend toward an inhibition of cell viability was shown for miR-222 in Ge970.2 and a significant inhibition was found in Ge518 (Supplementary FIG. 21).

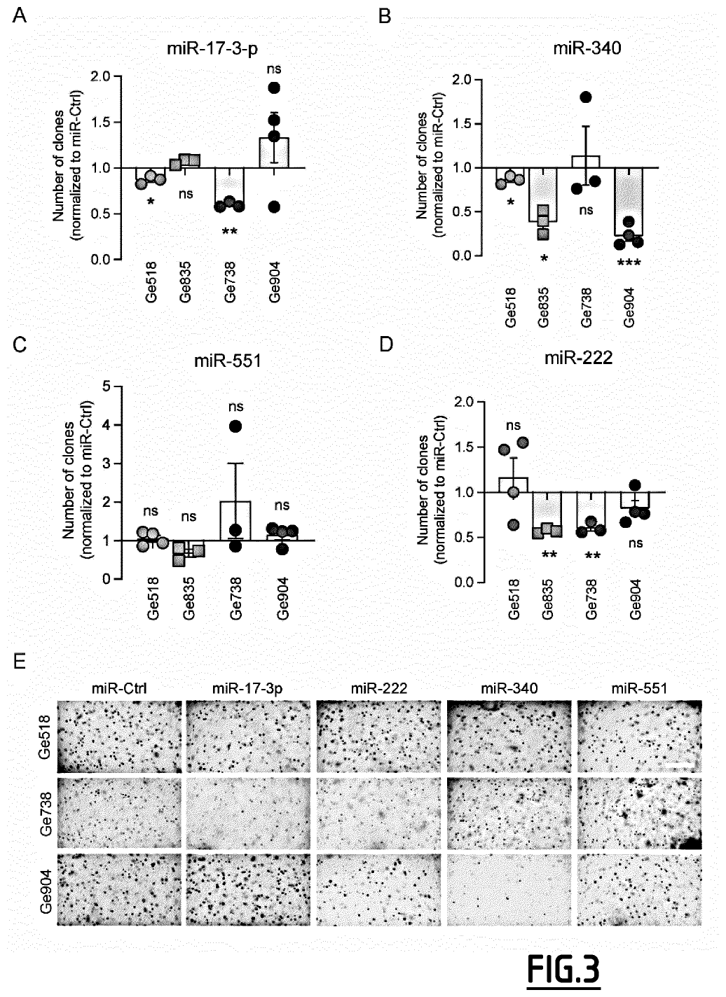

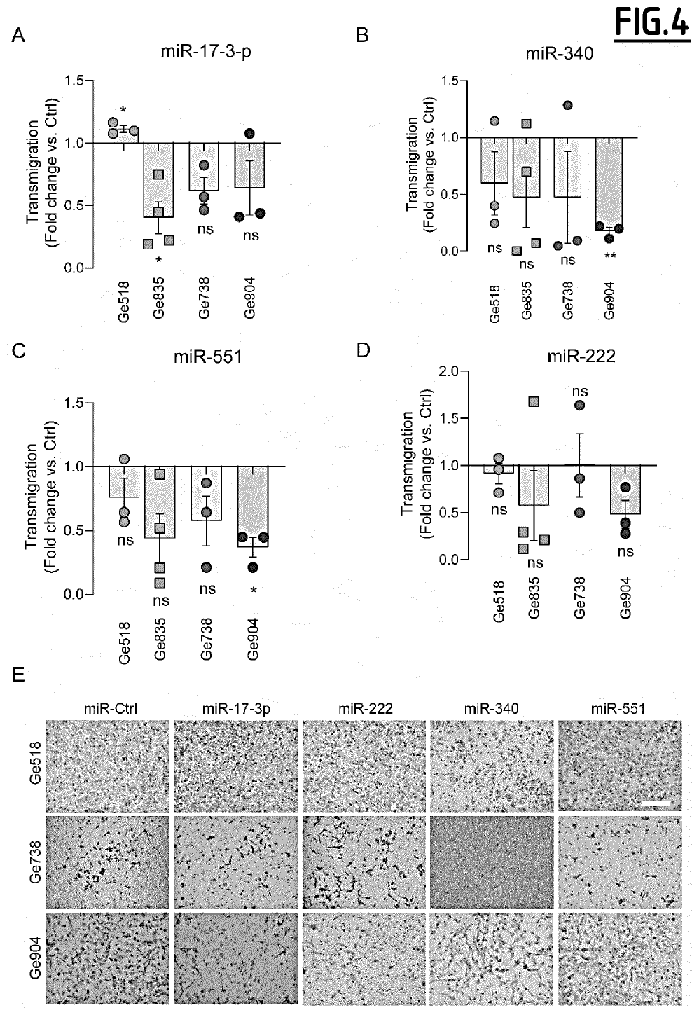

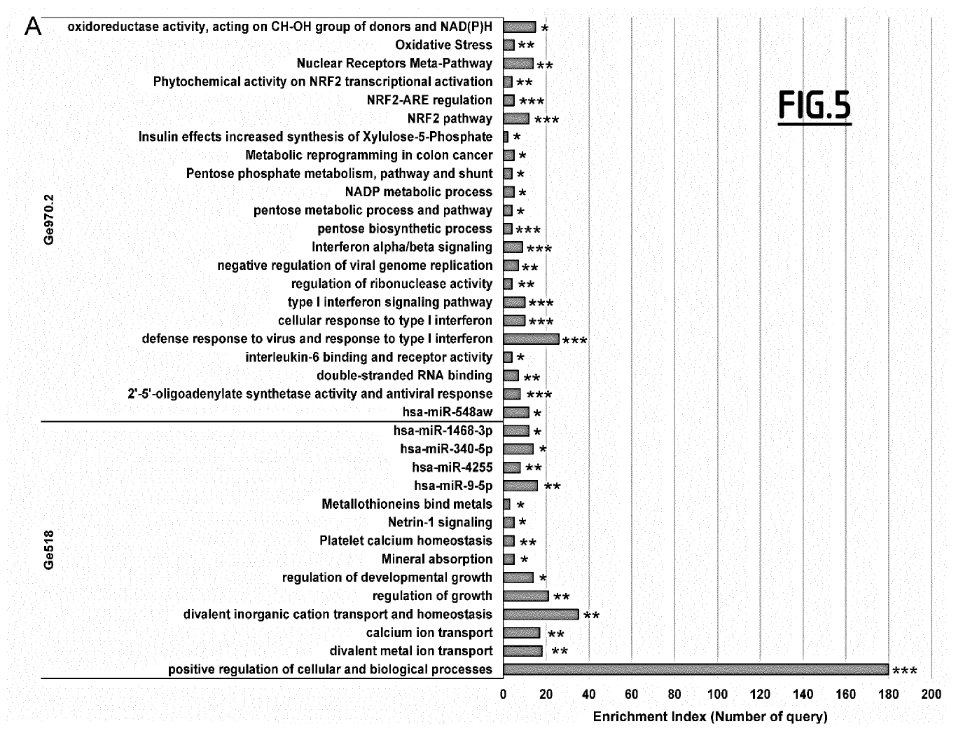

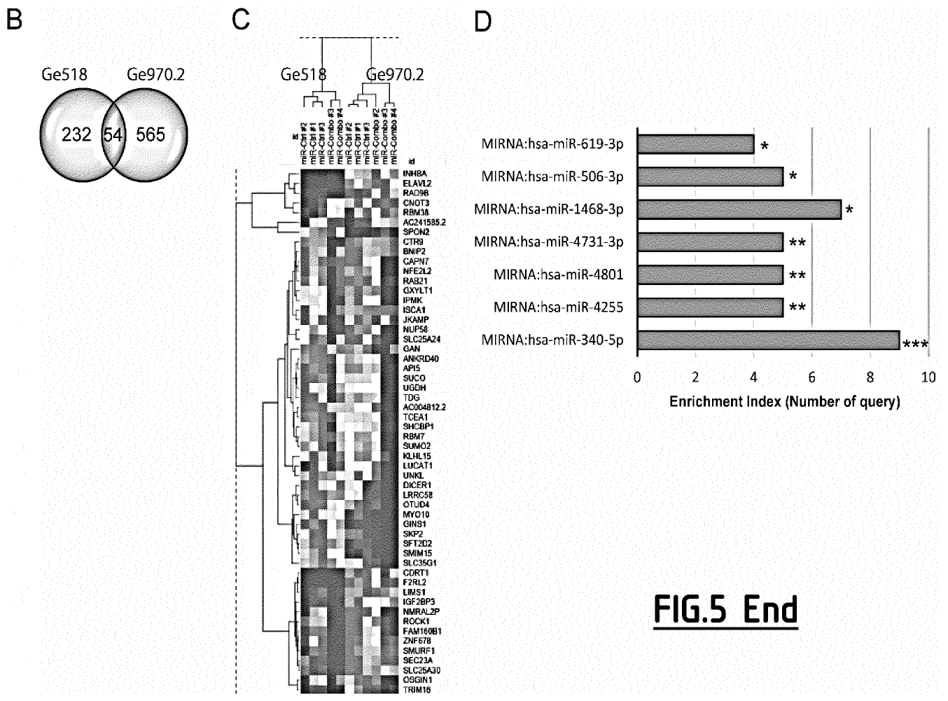

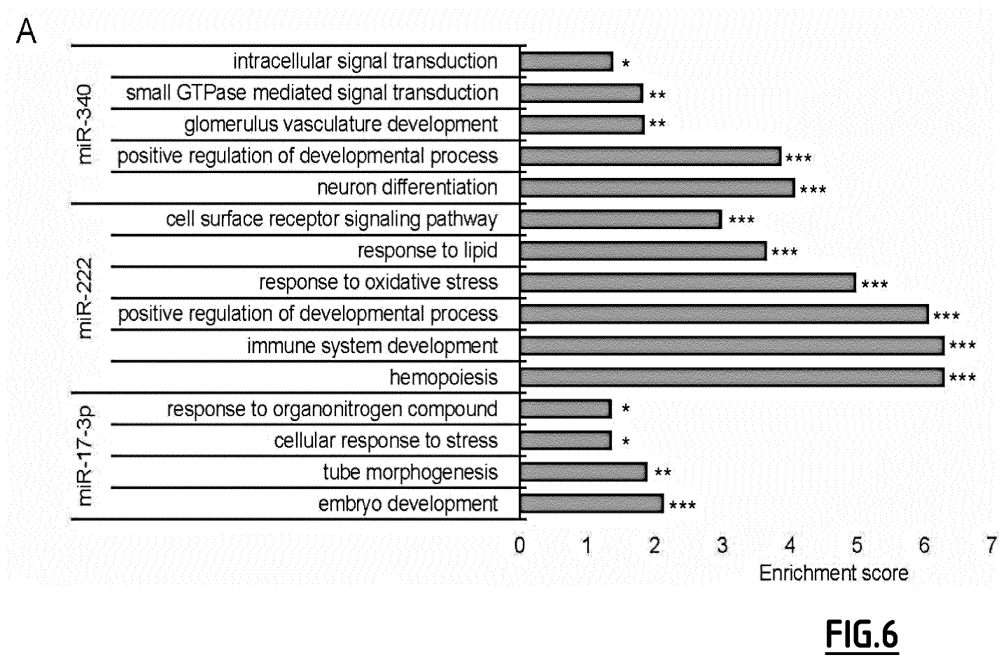

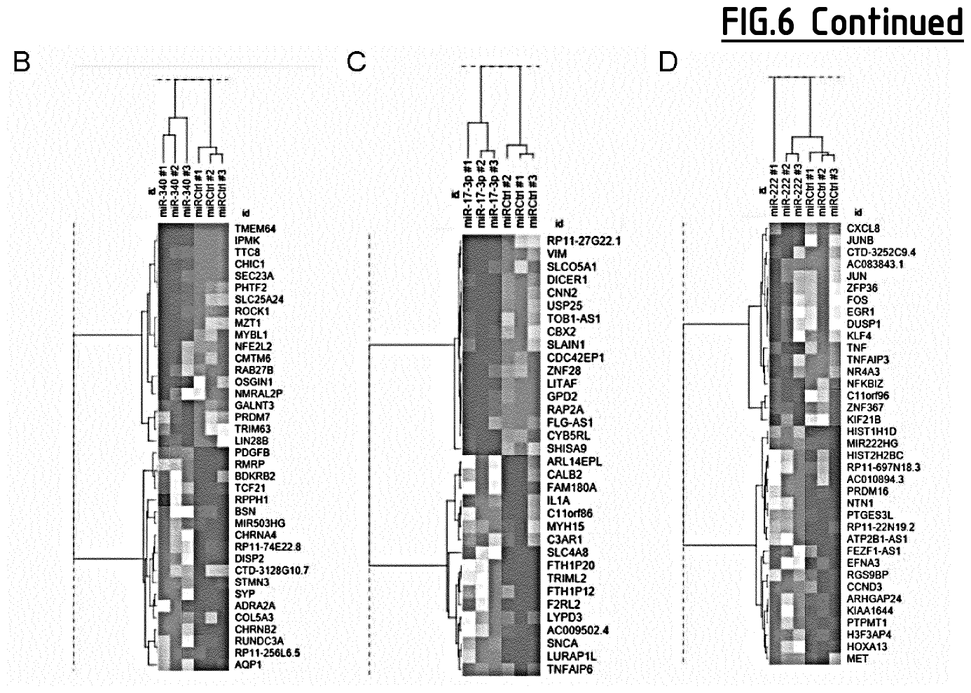

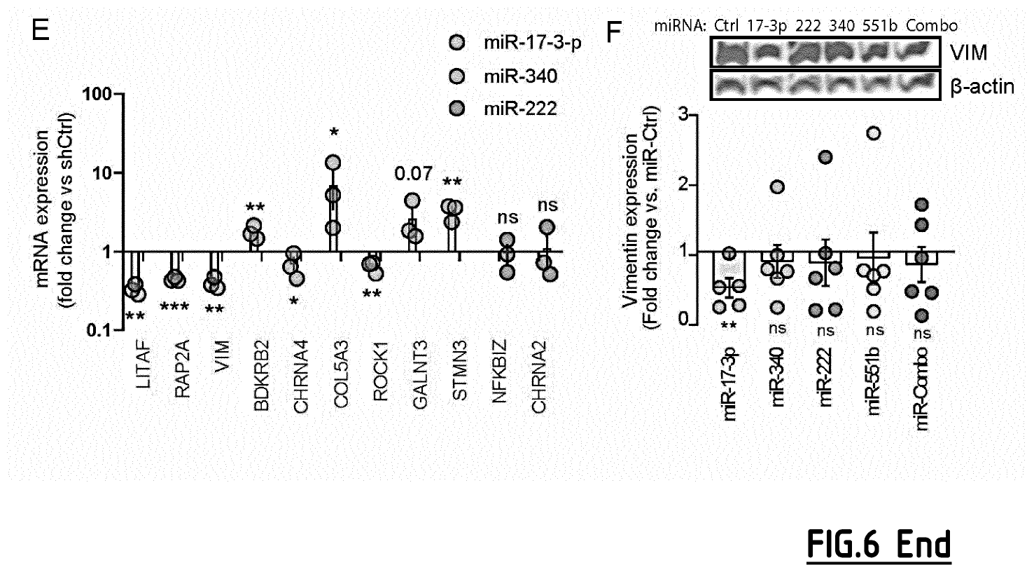

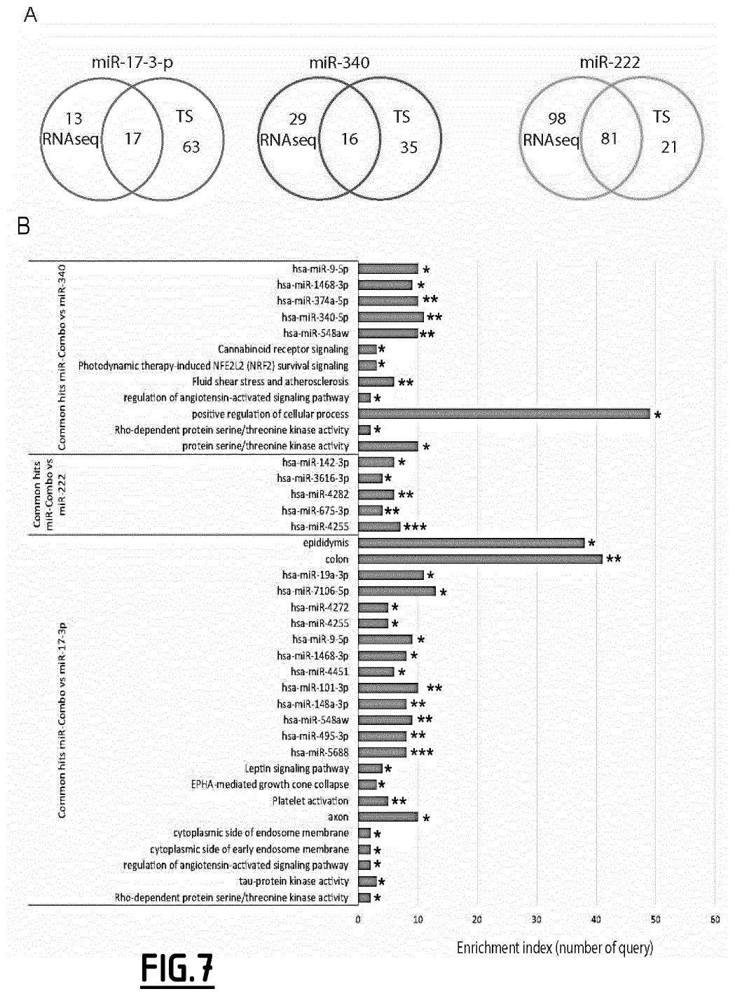

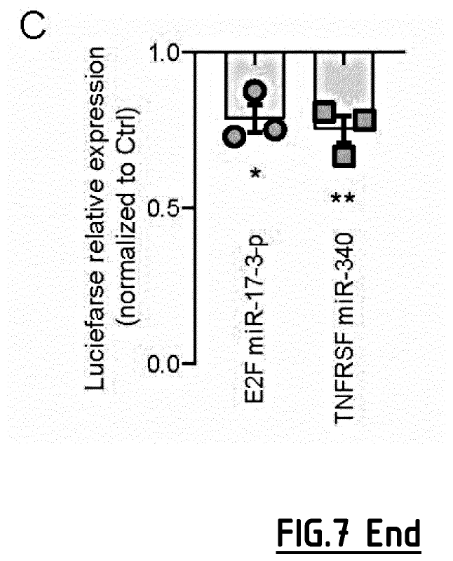

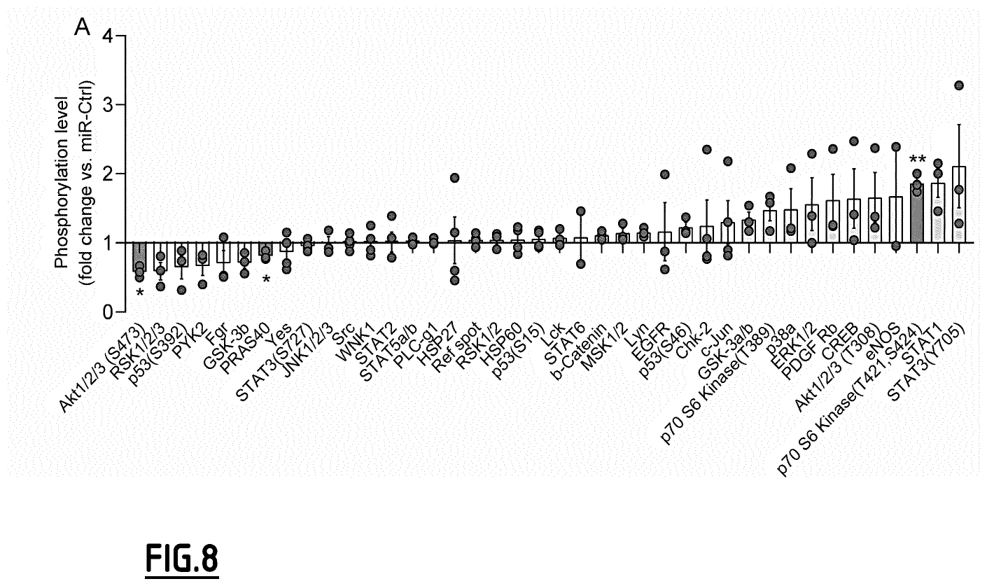

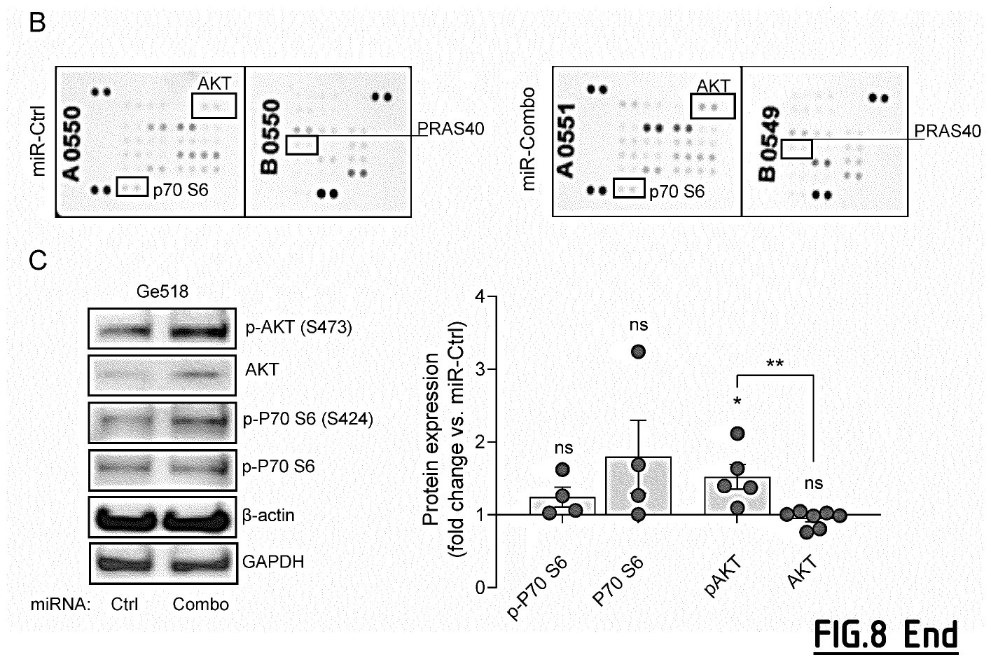

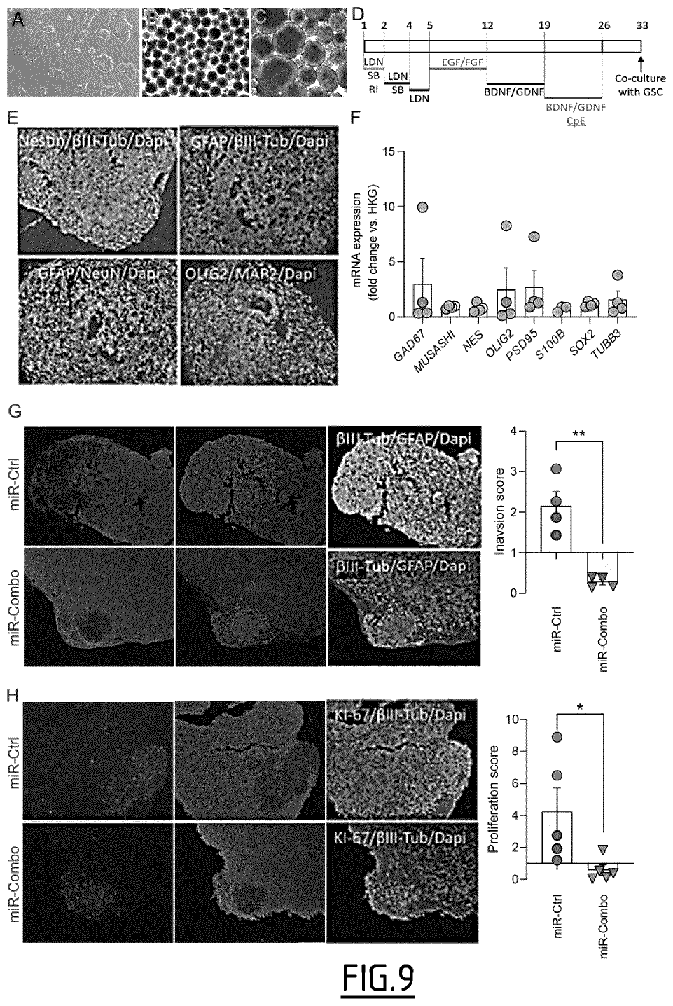

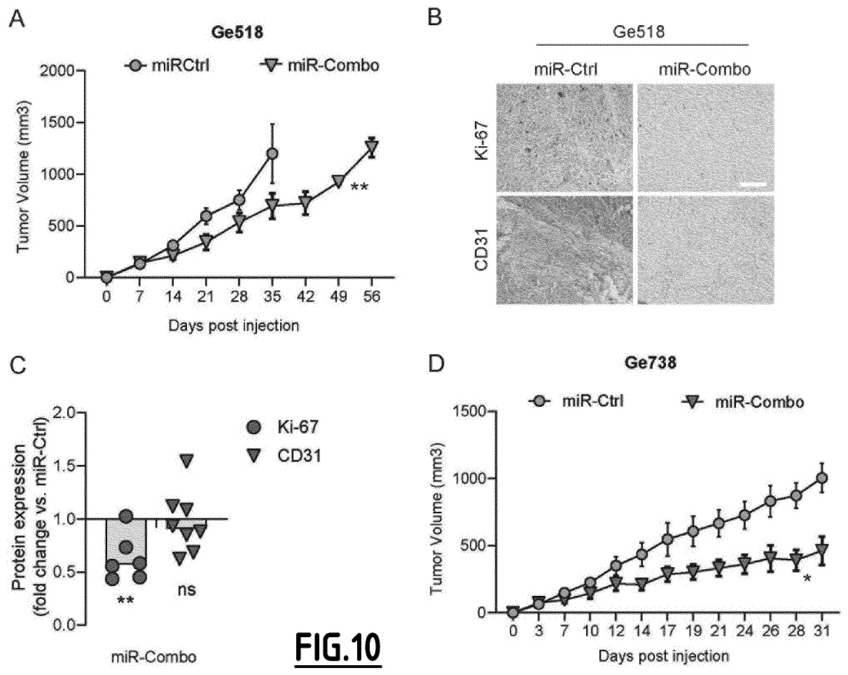

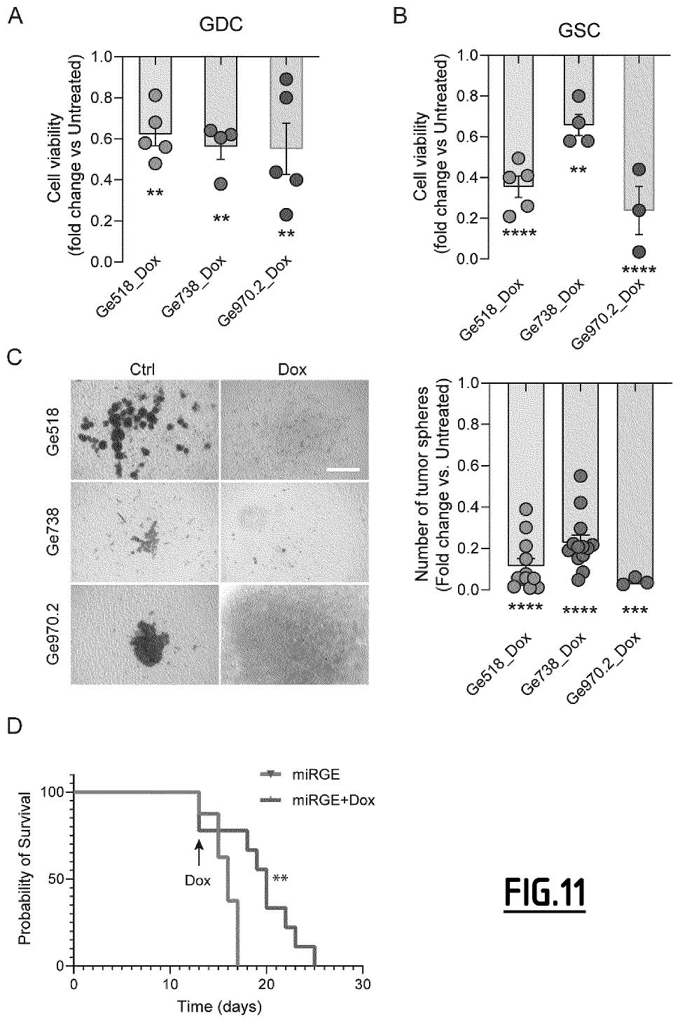

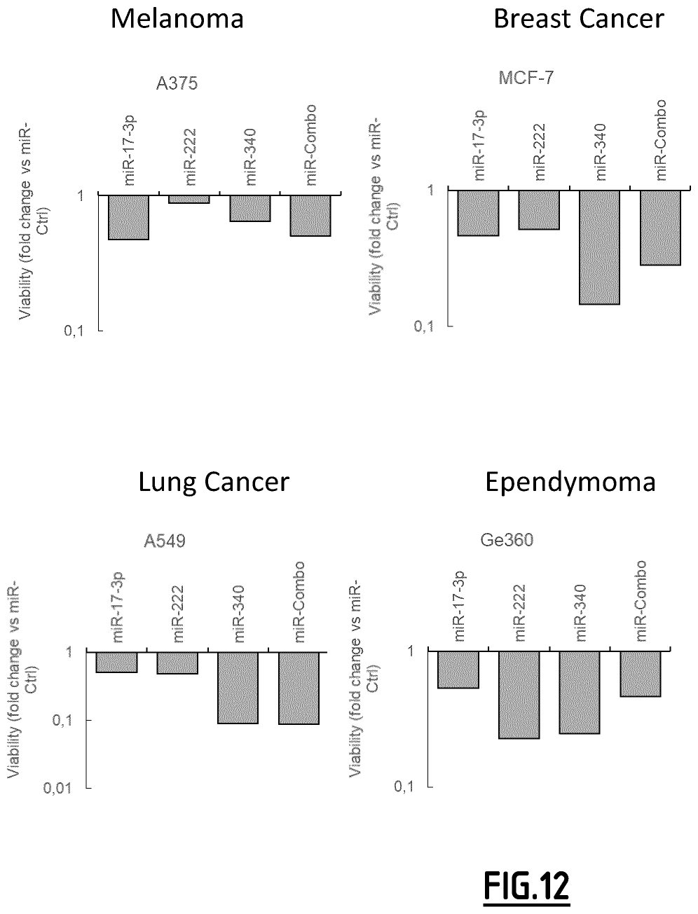

Then, to better characterize the biological effects of the miR-Combo, we also analyzed the transmigration and clonogenicity potential of GBM PDCs. The combinatorial modulation of miRNAs induced a significant decrease of cell clonogenicity and transmigration in all PDCs (FIG. 1C-D). Similarly, after the transfection of each miRNA separately, we showed that miR17-3p, miR-340, and miR-222 mediated most of the observed biological effects and has a different effect on different PDCs, highlighting the need to use them all in the combinatorial strategy (FIG. 3 and FIG. 4). Altogether, our data demonstrate that miR-17-3p, miR-340 and miR-222, inhibit GBM aggressiveness by affecting their survival, clonogenicity and transmigration capacity.

Modulation of miR-17-3p, miR-222 and miR-340 Regulates Gene Involved in Cell Viability

To understand the effect of the miR-Combo at the level of gene regulation, whole transcriptome analysis was performed in both Ge518, and Ge970.2 PDCs transfected with the miR-Combo compared to the non-targeting scrambled control (FIG. 5A). After bulk mRNA extraction, RNASeq analysis was performed in three biological replicates for each of the cell lines. In Ge518 PDCs, the gene ontology enrichment analysis revealed expression of genes involved in three main families: cellular and biological processes (MT2A, CDKL5, CXCR4, MAPK14, PLXNA4, RAB21, TGFBR3, VGLL4), cation transport and homeostasis (CHRNB2, RRAD, CACNA1H, GPR35, P2RX5, ATP2A1), and regulation of growth (MAPK11, NRCAM, SHTN1, SMURFI, CDKN1B, TNKS2, GDF5, VGLL4) (FIG. 5A). The analysis of miRTarBase, a database of miRNA-target interactions, via g:Profiler 22, showed an enrichment for miR-340-5p (ROCK1, LIMS1, ANKRD40, SKP2, FRS2) and miR-9-5p (SFT2D2, DICER1, IGF2BP3, SPON2, CXCR4, SERPINH1, RAP2A) target genes. In Ge970.2 PDCs, a strong and significant enrichment for gene involved in the antiviral response and type I interferon signaling was found (OAS-1, -2, -3, -L, MX-1, -2, IFI44L) (FIG. 5A). Moreover, genes involved in metabolic reprogramming and the pentose phosphase process and pathway (TKT, G6PD, PGD, TALDO1) were found enriched as well as NRF2 pathway (NFE2L2, GCLC, NQO1, YES1), and genes involved in oxidative stress. Altogether, this analysis revealed different cellular response to miR-Combo in the two models. To identify common hits, we compared the transcriptional profiling of both PDCs, Ge518 and Ge970.2, and found 54 common genes (FIG. 5B). The hierarchical clustering data grouped the samples by cell line first, confirming the significant level of heterogeneity of GBM lines, as, we and others have already described (FIG. 5C) (Calvo Tardon et al. (2020). An Experimentally Defined Hypoxia Gene Signature in Glioblastoma and Its Modulation by Metformin. Biology (Basel) 9; Sottoriva, A, et al. (2013). Intratumor heterogeneity in human glioblastoma reflects cancer evolutionary dynamics. Proc Natl Acad Sci USA 110: 4009-4014; Patel, et al. (2014). Singlecell RNA-seq highlights intratumoral heterogeneity in primary glioblastoma. Science 344: 1396-1401). Then, the data were grouped by conditions, miR-Combo vs miR-Ctrl. For both PDCs, we observed a significant decrease of several genes already found to be involved in GBM aggressiveness such as ROCK1 18, 26, LIMS1 18, SKP2 27, NFE2L2 28, RAB2129. In contrast, the expression of ELAVL2, RAD9B and CNOT3, already identified as tumor suppressor (Nord, H, et al. (2009). Characterization of novel and complex genomic aberrations in glioblastoma using a 32K BAC array. Neuro Oncol 11: 803-818), and inhibitors of cell cycle progression 31, respectively, were upregulated in the miR-Combo condition (FIG. 5C). Finally, the gene ontology enrichment analysis identified an enrichment for several miRNA such as miR-340-5p and miR-4255 (KLHL15, LRRC58, OTUD4, SEC23A, SUCO) (FIG. 5D). Collectively, our data highlighted a subset of critical genes that are modulated in GBM PDCs following miR-Combo transfection.

Then, to investigate the effect of each miRNA separately, the whole transcriptome analysis was performed in the Ge518 cell line transfected with miR-17-3p, and miR-340 mimics and miR-222 antagomir. The gene ontology enrichment analysis revealed expression of genes involved in cellular response to stress as a common pathway for miR-17-3p and miR-222 (FIG. 6A). Moreover, we found genes involved in signal transduction for miR-340 and miR-222. For miR-340, as previously shown (Cosset et al. (2016). Human tissue engineering allows the identification of active miRNA regulators of glioblastoma aggressiveness. Biomaterials 107: 74-87; Fiore, D, et al. (2016). miR-340 predicts glioblastoma survival and modulates key cancer hallmarks through down-regulation of NRAS. Oncotarget 7: 19531-19547), in a different cellular context, we confirmed ROCK1 and LIMS1 as miR-340 target genes (FIG. 6A-B). Moreover, we detected genes involved in neuron development and differentiation, small GTPase mediated signal transduction as well as cell surface receptor signaling pathway. For miR17-3p, we found three gene families involved in morphogenesis and embryo development, cellular response to stress and response to organonitrogen compound (FIG. 6A, C). For miR-222, multiple families of genes were found to be dysregulated such as response to oxidative stress/reactive oxygen species/drug, hematopoiesis and immune system development, and regulation of transcription by RNA polymerase (FIG. 6A, D). To confirm these findings, a set of representative genes from each functional family was selected and was validated by quantitative RT-PCR and/or western blots (FIG. 6E-F). Furthermore, the expression of several of

these genes, such as NFE2L2, COL5A3 and BDKRB2 for miR-340, ZFP36, NFKBIZ, and NTN1 for miR-222, and LITAF, and F2RL2 for miR-17-3P is correlated with poor survival (Table 4).

TABLE 4
P Values of the Kaplan-Meier analysis of the TCGA, Rembrant,
and Gravendeel datasets for the listed genes.

| | Log-rank pValue | | |
|---|---|---|---|
| Name | TCGA | Rembrandt | Gravendeel |
| NFE2L2 | 0.0015 | 0.0064 | 0.0109 |
| COL5A3 | 0.0757 | <0.01 | 0.0005 |
| ZFP36 | 0.0974 | 0.0162 | 0.0006 |
| NFKBIZ | 0.0637 | <0.01 | <0.01 |
| SHISA9 | 0.0232 | 0.0013 | 9.77E−02 |
| NTN1 | 0.0077 | 0.2524 | 0.6112 |
| LITAF | 0.0032 | <0.01 | <0.01 |
| F2RL2 | 0.0257 | <0.01 | 0.0036 |
| BDKRB2 | 0.164 | 0.0129 | 0.0012 |

Of note, when we compared the RNASeq analysis with the predicted miRNA target genes, we found a relatively short number of common hits (FIG. 7A). The RNASeq of the miR-Combo vs miR-Control (miR-Ctrl) with the RNASeq performed for each miRNA separately showed that each miRNA contributes to the gene modulation mediated by the combinatorial strategy (FIG. 7B).

To validate miRNA targeting of the 3′UTR, luciferase reporter constructs bearing the full-length of E2F-3′UTR and TNFRSF-3′UTR were used and transiently co-transfected in the Ge904 model with the miR-Ctrl vs miR-17-3p and miR-340, respectively (FIG. 7C). The ectopic expression of miR-17-3p and miR-340 resulted in a 1.5 fold decrease of luciferase activity in the cells containing the reporter constructs compared to their respective controls, providing evidence that the identified miRNAs act as active miRNAs.

Mechanistically, to identify and understand how the miR-Combo can modulate downstream signaling, we analyzed the phospho-kinase activity with the human phospho-proteome array (FIG. 8A-B). After transfection, the Ge518 PDCs were submitted to the proteome profiler array, which detects phosphorylated proteins in the cell lysates. We identified a significant activation of p70 S6 kinase as well as a significant inhibition of AKT and PRAS40 activities (FIG. 8A). The ribosomal S6 protein kinase, p70 S6 kinase, a downstream substrate of mTOR, is known for its role in controlling cell-cycle progression and cell survival. Moreover, PRAS40 is known to inhibit mammalian target of rapamycin C1 (mTORC1) activity. Indeed, by binding to Raptor, PRAS40 competes with the mTOR substrates, 4E-BP1 and p70S6K. These data are consistent with the inhibition of cell survival observed when the cells are transfected with miR-Combo. To confirm these results, we analyzed the expression and activation of AKT and P70 S6 kinase by immunoblotting (FIG. 8C). Even if, we observed a trend toward an increase of P70 S6 phosphorylation, we did not observe a significant modulation of its activity. Interestingly, we observed a trend toward an increase of the pool of P70 S6 kinase in the combo compared to the control (FIG. 8C).

In contrast, no difference was found between the combo and the control for AKT total and a significant increase in AKT activation was observed in the combo, confirming the human phospho-proteome analysis.

The Combinatorial Modulation of miR-17-3p, miR-222, and miR-340 Inhibits GBM PDC Invasion in Neural Organoid

We have recently developed an in vitro tissue engineering approach to generate 3D human brain-like from tissue from pluripotent stem cell (PSCs) towards the astro-neural fate (Cosset, et al. (2016). Human tissue engineering allows the identification of active miRNA regulators of glioblastoma aggressiveness. Biomaterials 107: 74-87; Cosset, et al. (2019). Human Neural Organoids for Studying Brain Cancer and Neurodegenerative Diseases. J Vis Exp.). Previously, we demonstrated that GBM cells proliferate and develop into brain-like tissues, generating a mixed tissue mimicking some critical and important features of the in vivo host/tumor interaction (Cosset, et al. (2016). Human tissue engineering allows the identification of active miRNA regulators of glioblastoma aggressiveness. Biomaterials 107: 74-87; Nayernia, et al. (2013). The relationship between brain tumor cell invasion of engineered neural tissues and in vivo features of glioblastoma. Biomaterials 34: 8279-8290.). Therefore, to study the inhibitory potential of the miRNA combo (miR-Combo) in 3D, we used this protocol to generate neural organoids (FIG. 9A-D). The characterization of the neural organoids showed an expression in neural (βIII-Tubulin (TUBB3), MAP2 and NeuN), astrocytic (GFAP, S1 00B) and oligodendrocytic (OLIG2) markers, at the mRNA and protein levels, confirming the neural maturation (FIG. 9E-F). Because GBM stem cell (GSC) represent the most aggressive and drug resistant cells within the tumor, and display self-renewal and tumor-initiation properties, we decided to use them as a model to confirm the miRNA multi-targeting strategy (Singh, et al. (2004). Identification of human brain tumour initiating cells. Nature 432: 396-401; Bao et al. (2006). Glioma stem cells promote radioresistance by preferential activation of the DNA damage response. Nature 444: 756-760). We co-cultured GSC Ge904 with a neural organoid for 24 hours and thereafter transfected with the miR-Combo (FIG. 9G-H). Four days post-transfection, for GSC Ge904, we assessed cell invasion and proliferation with the respective markers GFAP and Ki-67, and the neural organoid, we used βIII-Tubulin (FIG. 9G-H). We observed invasion of the GSCs into the neural organoid in the miR-Ctrl condition while the cells transfected with miR-Combo remained compact and not invasive (FIG. 9G). In miR-Control condition, the GSCs are bigger as they proliferate whereas in the miR-Combo they are smaller as if they stop proliferating (FIG. 9H). Accordingly, we observed a stronger signal for Ki-67 with invasive single cells far away from their primary site. Collectively, these data indicate that miRNAs can penetrate the 3D co-culture and affect GBM cell behavior by inhibiting their proliferation and invasive capacity.

The Tumor Growth of GBM Xenografts is Reduced by miR-17-3p, miR-222 and miR-340 Combo Treatment

To translate our findings to the clinic, we sought to investigate the effects of the combinatorial treatment on Ge518 tumor growth in vivo. To this end, we subcutaneously injected Ge518 cells to the flanks of nude mice (FIG. 10A). Once the tumors reached 150 mm2, we treated the mice with miR-Combo. We showed a significant delay of tumor growth, indicating that miR-Combo efficiently affects GBM growth and tumorigenic capacity, as shown in vitro (FIG. 10A). We also found a significant decrease of Ki-67 expression, a marker of cell proliferation, in miR-Combo compared to the miR-Ctrl, confirming the effect of the combinatorial strategy on GBM cell proliferation (FIG. 10B-C). The analysis of tumor vascularization assessed by CD31 staining showed no significant differences but we could observe a trend towards a decrease of CD31 expression and smaller vessels (FIG. 10B-C). To confirm these results, we used another PDC model, Ge738, and showed a similar delay in tumor growth (FIG. 10D). Altogether, we showed that the combinatorial targeting therapy consisting of modulating miR-17-3p, miR-340 and miR-222 is efficient to inhibit GBM growth and tumorigenic capacity in vivo.

By using the well-described miRGE lentivector system (Myburgh, et al. (2014). Optimization of Critical Hairpin Features Allows miRNA-based Gene Knockdown Upon Singlecopy Transduction. Mol Ther Nucleic Acids 3: e207.), we simultaneously expressed miR-17-3p, and miR-340 mimics, and miR-222 antagomiR in GBM PDCs.

Then, with the Tet-On system, miRNA modulation was turned on by treating the GBM cells with doxycycline. As shown in FIG. 11A, doxycycline treatment induced a significant decrease of cell viability in both Ge518, Ge738, and Ge970.2 PDCs.

Then, we also sought to investigate the effect of miRGE on GSCs, an aggressive subset of the GBM tumors. Consistent with our results in PDCs transfected with miR-Combo, we also observed a significance decrease of PDCs bearing miRGE viability under doxycycline treatment (FIG. 11A). Similarly, we observed this effect of doxycycline treatment on miRGE GSC viability after three days, indicating the potential of this combinatorial strategy in both differentiated and stem cells (FIG. 11B). In line with these results, a significant inhibition of their tumorsphere-forming ability was shown under doxycycline treatment (FIG. 11C). More importantly, to evaluate the effect of these miRNAs on GSC tumorigenicity in vivo, we intracranially implanted a mixture of mesenchymal (Ge518), proneural (Ge738) and classical (Ge970.2) GSC transduced with the miRGE lentivector system (1:1:1) into the brain of immunodeficient mice (FIG. 11D). Indeed, a mixture of GSC from several subtypes mimics the in vivo context where multiple subtypes co-exist. To leverage our findings to the clinic, the administration of doxycycline to the drinking water was done only after 13 days when the first mice developed neurological symptoms. Here, we showed a significant delay of tumor growth in the doxycycline-treated group compared to the untreated control group (FIG. 11D). Altogether, these results highlight the clinical relevance of the combinatorial strategy as not only a relevant prognostic biomarker but also as druggable targets for the treatment of GBM, where a significant unmet need for therapies remains.

### Example 2

### Material and Methods

### Cell Transfection

The miR-17-3p, and miR-340-5p mimics, and miR-222-5p antagomir were transfected using lipofectamine RNAimax (Invitrogen), at a final concentration of 5 nM, according to the manufacturer's protocols. A non-targeting scrambled miRNA (Life Technologies) was used as control.

### Cell Viability Assay

Cell viability assay was performed by using CellTiter-Glo assay kit (Promega) according to the manufacturer's protocols.

### Results

Results are shown in FIG. 12.

1.-15. (canceled)

16. A method for preventing or treating cancer in a patient, comprising administering the patient with a combination of a miR-17mimic and a miR-340 mimic.

17. The method of claim 16, wherein the patient is further administered with a miR-222 antagomiR.

18. The method of claim 16, wherein the patient is further administered with a miR-221 antagomiR and/or a miR-551b mimic.

19. The method of claim 16, wherein the miR-17mimic is a miR-17-3p mimic.

20. The method of claim 16, wherein the miR-340 mimic is a miR-340-5p mimic.

21. The method of claim 17, wherein the miR-222 antagomiR is selected from miR-222-5p and miR-222-3p antogomiRs.

22. The method of claim 18, wherein the miR-221 antagomiR is selected from miR-221-3p and miR-221-5p antagomiRs and/or the miR-551b mimic is a miR-551b-5p mimic.

23. The method of claim 16, wherein the cancer is selected from the group consisting of brain tumors, medulloblastomas, retinoblastomas, schwannomas, neuroblastomas, melanomas, NSCLCC, pancreatic tumors, breast cancers, hepatocarcinomas, and nephroblastomas.

24. The method of claim 23, wherein the brain tumor is selected from the group consisting of astrocytomas, oligodendrogliomas, meningiomas, gliomas and glioblastomas.

25. The method of claim 24, wherein the cancer is a glioblastoma.

26. The method of claim 25, wherein the glioblastoma is of the following subtypes: mesenchymal, proneural, neural, and classical.

27. The method of claim 16, wherein the miR-17mimic and the miR-340 mimic are administered sequentially.

28. The method of claim 16, wherein the miR-17mimic and the miR-340 mimic are administered concomitantly.

29. The method of claim 16, wherein said combination is formulated into a pharmaceutical composition.

30. The method of claim 29, wherein said pharmaceutical composition is administered, intravenously, orally or subcutaneously.
